SEQUENCE LISTING (2 sequences)

SEQ ID NO: 1 - RNA, 102 nt, Artificial Sequence
  sgRNA scaffold
nnnnnnnnnnnnnnnnnnnnguuuuagagcuagaaauagcaaguuaaaauaaggcuaguccguuaucaacuugaaaaaguggcaccgagucggugcuuuuuu

SEQ ID NO: 2 - RNA, 112 nt, Artificial Sequence
  sgRNA scaffold
nnnnnnnnnnnnnnnnnnnnguuuaagagcuaugcuggaaacagcauagcaaguuuaaauaaggcuaguccguuaucaacuugaaaaaguggcaccgagucggugcuuuuuu

(19) United States
(12) Patent Application Publication
(10) Pub. No.: US 2015/0315252 A1
(43) Pub. Date: Nov. 5, 2015

(54) PROTEIN ENRICHED MICROVESICLES AND METHODS OF MAKING AND USING THE SAME

(71) Applicant: Clontech Laboratories, Inc., Mountain View, CA (US)

(72) Inventors: Michael Haugwitz, Belmont, CA (US); Thomas Patrick Quinn, Sunnyvale, CA (US); Montserrat Morrell, Menlo Park, CA (US); Andrew Alan Farmer, Los Altos, CA (US)

(21) Appl. No.: 14/737,423

(22) Filed: Jun. 11, 2015

Related U.S. Application Data
(63) Continuation-in-part of application No. 14/278,714, filed on May 15, 2014.
(60) Provisional application No. 61/872,115, filed on Aug. 30, 2013.
(60) Provisional application No. 61/833,880, filed on Jun. 11, 2013.
(60) Provisional application No. 62/011,528, filed on Jun. 12, 2014.

(51) Int. Cl. C07K 14/435

(52) U.S. Cl. CPC C07K 14/435

(57) ABSTRACT

The present invention relates to a microvesicle comprising: (i) a membrane-associated protein comprising at least one first dimerization domain, (ii) a carrier protein comprising at least one second dimerization domain, and (iii) a solute that binds to the carrier protein, wherein the solute is selected from the group of: DNA, RNA, protein, carbohydrate, ribosomes, mitochondria, and small molecules. Also provided are cells, reagents and kits that find use in making the microvesicles, as well as methods of using the microvesicles, e.g., in research and therapeutic applications

This application is a continuation-in-part of U.S. application Ser. No. 14/278,714, filed May 15, 2014, which claims the benefit of U.S. Provisional Application Nos. 61/872,115, filed Aug. 30, 2013 and 61/833,880, filed Jun. 11, 2013. This application further claims the benefit of U.S. Provisional Application No. 62/011,528, filed Jun. 12, 2014. The contents of the above-identified applications are incorporated herein by reference in their entireties.

### BACKGROUND

Cell modification finds use in a variety of different applications, including research, diagnostic and therapeutic applications. Cell modification may be achieved using a number of different approaches, including the introduction of exogenous nucleic acids and/or proteins into a cell.

Protein delivery, which is known in the art as protein transduction, is the process by which a peptide or protein motif is delivered across the plasma membrane into the cell. Protein delivery methods include micro-injection and electroporation. Protein delivery methods also include: transfection by forming complexes with lipid-based reagents; transfection by forming complexes with polymer or peptide based reagents; direct addition through inclusion of a peptide transduction domain (PTD) to the protein of interest; virus like particle mediated introduction; and exosome mediated protein introduction.

One drawback of the current methods of protein delivery is the requirement to produce a stock of purified protein for transfection into the desired target cell. Standard methods for the production of recombinant protein can present issues with solubility, yield, correct folding and post-translational modifications. These methods also do not allow for the delivery of recombinant membrane proteins. Many of these factors are important because they directly relate to the activity of the protein to be transfected. The activity of the protein has the highest priority for direct delivery so that the delivered protein will exert an effect on a cell.

Another drawback to the current methods lies in the delivery itself. Both the lipid and polymer/peptide based transfection methods have issues with protein specific packaging efficiency due to unfavorable charge differences as well as inefficient delivery and toxicity. Electroporation also has been shown to have issues with toxicity, high level of inconsistency and a lack of control over the protein amount delivered. Inclusion of a PTD is known to cause aggregation and precipitation which can adversely affect delivery efficiency. Lastly, delivery of proteins in virus like particles (VLPs) requires that immune response-generating viral capsid proteins are used for packaging.

### BRIEF DESCRIPTION OF THE FIGURES

FIG. 1A is a schematic showing the workflow for producing microvesicles of the first aspect of the invention. FIGS. 1B-1D show the vector maps of the Cas9 microvesicle packaging mix (pDmrC Cas9, 1B; pCherry Picker DmrA, 1C; and pVSV-G, 1D)

FIG. 2A illustrates the packaging of a cargo protein of interest into microvesicles of the invention in the presence of a dimer-/multimer-ization inducing compound (A/C heterodimerizer). FIG. 2B Is a schematic illustrating the packaging of a cargo protein of interest into microvesicles of the invention in the absence of a dimer-/multimer-ization inducing compound.

FIG. 3 is a western blot showing how active packaging of a protein of interest into microvesicles of the invention via compound-induced dimerization/multimerization increases the amount of the protein of interest that can be packaged.

FIG. 4A provides a schematic for a genomically integrated LacZ reporter cassette in which expression of the LacZ reporter is blocked by the presence of a translational stop cassette, flanked by LoxP sites. When Cre is provided to the cells, for example via microvesicles of the invention, the Cre protein will induce removal of the stop cassette via recombination between the flanking LoxP sites, thus allowing expression of the LacZ reporter; turning the cells blue in the presence of appropriate substrate. FIG. 4B shows that rhabdomyosarcoma cell line harboring an integrated LacZ reporter cassette as described was treated with Cre containing microvesicles of the invention that had been produced either in the presence or absence of the dimer-/multimer-ization agent. Only in the case of cells treated with microvesicles made in the presence of the dimer-/multimer-ization agent has sufficient Cre present to cause activation of the LacZ cassette, allowing the cells to turn blue (see bottom panel).

FIG. 5A is a schematic outlining a method for readily screening targeted knockout induced by Cas9 using a cell line engineered to express a single copy of an AcGFP expression cassette. Ordinarily, this cell line will express the AcGFP fluorescent protein and thus display a green fluorescense signal by flowcytometry. When the cell contains a Cas9/sgRNA complex targeting the AcGFP reporter, this will cause a frameshift in the AcGFP cassette, resulting in a loss of expression and hence loss of fluorescence. FIG. 5B provides example flow cytometry profiles of cells either expressing AcGFP or not, showing how the difference in expression can be detected by flow cytometry.

FIG. 6 is a schematic illustrating a gene targeting experiment in which the sgRNA and Cas9 are provided to the target cell separately. While the sgRNA is provided via an expression plasmid, the Cas9 is provided using microvesicles of the invention packaged in the presence of the A/C heterodimerizer.

FIG. 7 shows the results of a flow cytometry analysis of an AcGFP reporter cell line before and after delivery of an sgRNA targeting AcGFP, wherein the sgRNA is provided via plasmid transfection, and Cas9-NLS-DmrC protein is provided via microvesicles.

FIG. 8A provides the nucleotide sequence (SEQ ID NO: 1) of one sgRNA scaffold of use in practicing the invention. FIG. 8B provides the nucleotide sequence (SEQ ID NO: 2) of a second sgRNA scaffold of use in practicing the invention. In both cases the string of 20 ‘N’s represent the variable guide sequence—specific to the target gene of interest.

FIG. 9 shows the knockout efficiencies obtained using Cas9 protein, delivered via the microvesicles of the invention, in conjunction with each of the two example sgRNA scaffolds as depicted in FIG. 8A and FIG. 8B.

FIG. 10 illustrates the packaging of an RNA/protein complex (Cas9/sgRNA) into microvesicles of the invention using the A/C dimer-/multimer-ization compound.

FIG. 11 illustrates the delivery of a Cas9/sgRNA complex via microvesicles of the invention into a target cell.

FIG. 12 shows the results of a flow cytometry analysis of an AcGFP-reporter cell line before (left panel) and after (right panel) delivery of a Cas9/sgRNA complex via microvesicles of the invention; demonstrating the effective knock-out of the AcGFP gene.

FIG. 13 shows an experiment whereby knockout of the endogenous CD81 gene in HeLa cells was obtained using microvesicles of the invention containing an Cas9/sgRNA complex, wherein the sgRNA is targeting the CD81 gene.

FIG. 14 illustrates the packaging of a RNA of interest containing a stem loop repeat specifically interacting with the MS2 coat protein. This cargo protein/RNA complex is packaged into microvesicles in the presence of a dimer-/multimer-ization inducing compound (A/C heterodimerizer).

### DETAILED DESCRIPTION OF THE INVENTION

Protein enriched microvesicles and methods of making and using the same are provided. Also provided are cells, reagents and kits that find use in making the microvesicles, as well as methods of using the microvesicles, e.g., in research and therapeutic applications.

The first aspect of the invention is directed to a microvesicle comprising: (i) a membrane-associated protein comprising at least one first dimerization domain, and (ii) a nuclease protein comprising at least one second dimerization domain. In a preferred embodiment, the nuclease is selected from the group consisting of a Cas protein, and Argonaute nuclease.

The present invention provides a nuclease-enriched microvesicles. By “nuclease-enriched microvesicle” is meant a non-cellular fusogenic structure that includes an amount of one or more nucleases inside a lipid bilayer envelope. As used herein, the term “fusogenic” refers to the property of the microvesicle which provides for the fusion of the membrane of the microvesicles to the membrane of the target cell. As the microvesicles are fusogenic, they are capable of fusion with the lipid bilayer membrane of a target cell to deliver their contents, including the nuclease protein(s), into the cell.

Membrane-Associated Protein that Includes a First Dimerization Domain Cells employed in methods of making microvesicles include a membrane-associated protein having a first dimerization domain. The cell may include any convenient membrane-associated protein that includes a first dimerization domain. A membrane-associated protein is a protein that is capable of stably associating with, e.g., via a binding interaction, the membrane of a microvesicle, where the membrane-associated protein, when associated with a microvesicle membrane, may be configured so that the dimerization domain contacts the cytosol of the microvesicle. Membrane-associated proteins may vary in size including peptides, ranging in some instances from 500 Da to 250 k Da, such as 10 k Da to 100 kDa and 12 k Da to 50 kDa.

The membrane-associated protein may be modified to include a single dimerization domain or two or more dimerization domains (e.g., as described in greater detail below). In some cases, two or more membrane-associated proteins may be included in the subject cells and microvesicles. Each of the two or more membrane-associated proteins may independently include a dimerization domain for forming a dimerized complex with a nuclease protein.

Membrane-associated proteins of interest include, but are not limited to, any protein having a domain that stably associates, e.g., binds to, integrates into, etc., a cell membrane (i.e., a membrane-association domain), where such domains may include myristoylated domains, farnesylated domains, transmembrane domains, and the like. For example, a protein can be localized at the plasma membrane via myristoylation. Specific membrane-associated proteins of interest include, but are not limited to: myristoylated proteins, e.g., p60 v-src and the like; farnesylated proteins, e.g., Ras, Rheb and CENP-E,F, proteins binding specific lipid bilayer components e.g. AnnexinV, by binding to phosphatidyl-serine, a lipid component of the cell membrane bilayer and the like; membrane anchor proteins; transmembrane proteins, e.g., transferrin receptors and portions thereof and CherryPicker (a transmembrane red fluorescent protein, Clontech); viral membrane fusion proteins, e.g., as described below, VSV-G, and the like.

Membrane-associated proteins of interest, in addition to including a membrane-association domain, also include a first dimerization domain. The first dimerization domain may vary widely, and may be a domain that directly binds to a second dimerization domain of a nuclease protein or binds to a second dimerization domain via a dimerization mediator, e.g., as described in greater detail below. A given membrane-associated protein may include a single type of a given domain (e.g., dimerization domain, membrane associated domain, etc.) or multiple copies of a given domain, e.g., 2 or more, 3 or more, etc.; and/or multiple different dimerization domains, as desired. Additional domains may be present in a given membrane associated protein molecule, e.g., linker domains, detection domains (e.g., fluorescent proteins, other enzymatic reporters such as Luciferase and the like), etc., as desired. In a given membrane associated protein, the membrane association domain and dimerization domain(s) may be heterologous to each other, such that they are not naturally associated with each other. As such, the membrane associated protein of such embodiments is not a naturally occurring protein.

The cell may include a single membrane-associated protein or two or more distinct membrane-associated proteins, e.g., where two or more distinct nuclease proteins are desired to be packaged into a microvesicle. As such, a microvesicle producing cell according to aspects of the invention may include a single membrane-associated protein or two or more different membrane-associated proteins of differing sequence, e.g., 3 or more, 4 or more 5 or more, etc., where in some instances the number of distinct membrane-associated proteins of differing sequence ranges from 1 to 10, such as 1 to 5, including 1 to 4.

### Nuclease Protein

The microvesicles described herein may include any desired nuclease. In some instances, the nuclease is a nucleic acid guided nuclease. As used herein, a “nucleic acid guided nuclease” is a nuclease that is guided to a target nucleic acid by a guide nucleic acid. The nucleic acid guided nuclease may have nuclease/cleavage activity (e.g., catalyzes the hydrolysis of a target nucleic acid (e.g., a target DNA, a target RNA, etc.) into two or more products.

In certain aspects, the nucleic acid guided nuclease includes a nucleic acid guide component and a nuclease component, where these two components are stably associated with each other. Any suitable nuclease component may be employed by a practitioner of the subject methods. The nuclease component may be a wild-type enzyme that exhibits nuclease activity, or a modified variant thereof that may or may not retain its nuclease activity. In other aspects, the nuclease component may be a non-nuclease protein operatively linked to a heterologous nuclease (or “cleavage”) domain, such that the protein is capable of cleaving the target nucleic acid by virtue of being linked to the nuclease domain. Such a strategy has been successfully employed to confer nuclease activity upon zinc finger and transcription-activator-like effector (TALE) proteins to generate zinc finger nucleases and TALENs, respectively, for genomic engineering purposes (see, e.g., Kim et al. (1996) PNAS 93(3):1156-1160, and US Patent Application Publication Numbers US2003/0232410, US2005/0208489, US2006/0188987, US2006/0063231, and US2011/0301073). According to certain embodiments, the nuclease domain is derived from an endonuclease. Endonucleases from which a nuclease/cleavage domain can be derived include, but are not limited to: a Cas nuclease (e.g., a Cas9 nuclease), an Argonaute nuclease (e.g., Tth Ago, mammalian Ago2, etc.), 51 Nuclease; mung bean nuclease; pancreatic DNase I; micrococcal nuclease; yeast HO endonuclease; a restriction endonuclease; a homing endonuclease; and the like; see also Mishra (Nucleases: Molecular Biology and Applications (2002) ISBN-10: 0471394610).

According to certain embodiments, the nucleic acid guided nuclease includes a CRISPR-associated (or “Cas”) nuclease. The CRISPR/Cas system is an RNA-mediated genome defense pathway in archaea and many bacteria having similarities to the eukaryotic RNA interference (RNAi) pathway. The pathway arises from two evolutionarily (and often physically) linked gene loci: the CRISPR (clustered regularly interspaced short palindromic repeats) locus, which encodes RNA components of the system; and the Cas (CRISPR-associated) locus, which encodes proteins.

There are three types of CRISPR/Cas systems which all incorporate RNAs and Cas proteins. The Type II CRISPR system carries out double-strand breaks in target DNA in four sequential steps. First, two non-coding RNAs (the pre-crRNA array and tracrRNA), are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer.

CRISPR systems Types I and III both have Cas endonucleases that process the pre-crRNAs, that, when fully processed into crRNAs, assemble a multi-Cas protein complex that is capable of cleaving nucleic acids that are complementary to the crRNA. In type II CRISPR/Cas systems, crRNAs are produced by a mechanism in which a trans-activating RNA (tracrRNA) complementary to repeat sequences in the pre-crRNA, triggers processing by a double strand-specific RNase III in the presence of the Cas9 protein. Cas9 is then able to cleave a target DNA that is complementary to the mature crRNA in a manner dependent upon base-pairing between the crRNA and the target DNA, and the presence of a short motif in the crRNA referred to as the PAM sequence (protospacer adjacent motif).

The requirement of a crRNA-tracrRNA complex can be avoided by use of an engineered fusion of crRNA and tracrRNA to form a “single-guide RNA” (sgRNA) that comprises the hairpin normally formed by the annealing of the crRNA and the tracrRNA. See, e.g., Jinek et al. (2012) Science 337:816-821; Mali et al. (2013) Science 339:823-826; and Jiang et al. (2013) Nature Biotechnology 31:233-239. The sgRNA guides Cas9 to cleave target DNA when a double-stranded RNA:DNA heterodimer forms between the Cas-associated RNAs and the target DNA. This system, including the Cas9 protein and an engineered sgRNA containing a PAM sequence, has been used for RNA guided genome editing with editing efficiencies similar to ZFNs and TALENs. See, e.g., Hwang et al. (2013) Nature Biotechnology 31 (3):227.

According to certain embodiments, the nuclease component of the nucleic acid guided nuclease is a CRISPR-associated protein, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof. In certain aspects, the nuclease component of the nucleic acid guided nuclease is Cas9. The Cas9 may be from any organism of interest, including but not limited to, Streptococcus pyogenes (“spCas9”, Uniprot Q99ZW2) having a PAM sequence of NGG; Neisseria meningitidis (“nmCas9”, Uniprot C6S593) having a PAM sequence of NNNNGATT; streptococcus thermophilus (“stCas9”, Uniprot Q5M542) having a PAM sequence of NNAGAA, and Treponema denticols (“tdCas9”, Uniprot M2B9U0) having a PAM sequence of NAAAAC. An example nucleic acid guided nuclease that includes a Cas9 nuclease and a sgRNA guide component, in which the sgRNA guide component is aligned with a complementary region of a generalized target nucleic acid.

In certain aspects, the nuclease component of the nucleic acid guided nuclease is an Argonaute (Ago) nuclease. Ago proteins are a family of evolutionarily conserved proteins central to the RNA interference (RNAi) platform and microRNA (miRNA) function and biogenesis. They are best known as core components of the RNA-induced silencing complex (RISC) required for small RNA-mediated gene regulatory mechanisms. In post-transcriptional gene silencing, Ago guided by a small RNA (e.g., siRNA, miRNA, piRNA, etc.) binds to the complementary transcripts via base-pairing and serve as platforms for recruiting proteins to facilitate gene silencing.

Mammals have eight Argonaute proteins, which are divided into two subfamilies: the Piwi clade and the Ago clade. Of the wild-type Ago proteins (Ago1-4, or EIF2C1-4), only Ago2 has endonuclease activity. The crystal structure of full-length human Ago2 (Uniprot Q9UKV8) has been solved. See, e.g., Elkayam et al. (2012) Cell 150(1):100-110. Similar to the bacteria counterpart, human Ago2 is a bilobular structure comprising the N-terminal (N), PAZ, MID, and PIWI domains. The PAZ domain anchors the 3′end of the small RNAs and is dispensable for the catalytic activity of Ago2. However, PAZ domain deletion disrupts the ability of the non-catalytic Agos to unwind small RNA duplex and to form functional RISC.

When the nuclease component of the nucleic acid guided nuclease is an Ago nuclease, the nuclease may be derived from any suitable organism, such as a prokaryotic or eukaryotic organism. In certain aspects, the Ago is a prokaryotic Ago. Prokaryotic Agos of interest include, but are not limited to, Thermus thermophiles Ago (“Tth Ago”). DNA-guided DNA interference in vivo using Tth Ago and 5′-phosphorylated DNA guides of from 13-25 nucleotides in length was recently described by Swarts et al. (2014) Nature 507:258-261.

In some instances, the nuclease protein (s) is an endogenous protein. In some instances, the nuclease protein is a heterologous protein. As used herein, the term “heterologous” means that the protein is not expressed from a gene naturally found in the genome of the cell used to produce the microvesicle. The nuclease protein may also be a mutant of the wild-type protein, such as a deletion mutant or a point mutant and may show a gain of function or loss of function for example a dominant negative mutant of the wild-type protein. Nuclease proteins may also be chimeras of one of more protein domains so as to generate a nuclease protein of novel function—similar to other chimeric proteins—e.g., a Tet Transactivator, which is a fusion of a tet repressor domain and a transactivation domain to create a novel transcriptional regulator or proteins obtained via domain swapping etc. In certain embodiments, the nuclease protein does not include any viral membrane fusion protein or any fragment of a viral membrane fusion protein or derivatives retaining fusogenic properties.

In some instances, the nuclease component may include a nuclear localization signal, NLS. A “nuclear localizing sequence” is an amino acid sequence which acts like a ‘tag’ on the exposed surface of a protein. This sequence is used to target the protein to the cell nucleus through the Nuclear Pore Complex and to direct a newly synthesized protein into the nucleus via its recognition by cytosolic nuclear transport receptors. Typically, this signal consists of one or more short sequences of positively charged lysines or arginines. Different nuclear localized proteins may share the same NLS. An NLS has the opposite function of a nuclear export signal, which targets proteins out of the nucleus. Functional assays to determine whether a protein domain is capable of acting as a nuclear localizing sequence are known to the person skilled in the art. For example, a protein that yields a detectable signal, e.g. a fluorescent signal, such as green fluorescent protein and variants thereof, may be expressed within a cell. In one experiment, said protein is expressed without the potential NLS. In another experiment, said protein is expressed in a fusion protein with the potential NLS. The subcellular localization of the proteins can be observed by using a microscope. If the fusion protein comprising the potential NLS is present in the nucleus to a higher degree as compared the protein lacking the potential NLS, the potential NLS is indeed capable of acting as an NLS. Any convenient NLS may be employed which mediates nuclear transport into the nucleus, wherein deletion of the NLS prevents nuclear transport. In some embodiments, a NLS is a highly cationic peptide. Any convenient NLS sequence may be employed, including but not limited to, SV40 virus T-antigen. NLSs known in the art include, but are not limited to those discussed in Cokol et al., 2000, EMBO Reports, 1(5):411-415, Boulikas, T., 1993, Crit. Rev. Eukaryot. Gene Expr., 3:193-227, Collas, P. et al., 1996, Transgenic Research, 5: 451-458, Collas and Alestrom, 1997, Biochem. Cell Biol. 75: 633-640, Collas and Alestrom, 1998, Transgenic Resarch, 7: 303-309, Collas and Alestrom, 1996, Mol. Reprod. Devel., 45:431-438, all of which disclosures of NLSs therein are incorporated by reference herein.

The cell may include a single nuclease protein or two or more distinct nuclease proteins of differing sequence which are desired to be packaged into a microvesicle. As such, a microvesicle producing cell according to aspects of the invention may include a single nuclease protein or two or more distinct nuclease proteins of differing sequence, e.g., 3 or more, 4 or more 5 or more, etc., where in some instances the number of distinct nuclease proteins ranges from 1 to 10, such as 1 to 5, including 1 to 4.

Nuclease proteins according to the embodiments of the invention include a second dimerization domain. The second dimerization domain may vary widely, where the second dimerization domain is a domain that dimerizes (e.g., stably associates with, such as by non-covalent bonding interaction, either directly or through a mediator) with the first dimerization domain of the membrane associated protein, either directly or through a dimerization mediator, e.g., as described in greater detail below. A given nuclease protein may include a single type of a given domain (e.g., dimerization domain) or multiple copies of a given domain, e.g., 2 or more, 3 or more, etc. Additional domains may be present in a given nuclease protein molecule, e.g., linker domains, etc., as desired. In a given nuclease protein, the protein domain and dimerization domain(s) may be heterologous to each other, such that they are not naturally associated with each other. As such, the nuclease protein of such embodiments is not a naturally occurring protein.

### Dimerization Domains

In one embodiment, the membrane-associated protein comprising at least one first dimerization domain, and the nuclease protein comprising at least one second dimerization domain, are bound to each other through the first and the second dimerization domain and form a multimerized complex. In the multimer complex, in general n=2-10, preferably n=2-3, 2-4, or 2-5. For example, the multimer is a dimer, trimer, or a tetramer.

The dimerization domains of the membrane-associated and nuclease proteins may vary, where these dimerization domains may be configured to bind directly to each other or through a dimerization mediator, e.g., as described in greater detail below. Since the membrane-associated and nuclease protein each include both a membrane-associated domain or nuclease protein domain, respectively, and a dimerization domain, they may be viewed as chimeric proteins or fusion proteins having at least two distinct heterologous domains which are stably associated with each other. By “heterologous”, it is meant that the at least two distinct domains of these chimeric proteins do not naturally occur in the same molecule. As such, these chimeric proteins are composed of at least two distinct domains of different origin. As the two domains of these proteins are stably associated with each other, they do not dissociate from each other under cellular conditions, e.g., conditions at the surface of a cell, conditions inside of a cell, etc. In a given chimeric or fusion protein, the two domains may be associated with each other directly or via an amino acid linker, as desired. An amino acid linker may have any convenient amino acid sequence and length.

With respect to the dimerization domains, these domains are domains that participate in a binding event, either directly or via a dimerization mediator, where the binding event results in production of the desired multimeric, e.g., dimeric, complex of the membrane associated and nuclease proteins. As such, the first and second dimerization domains are domains that participate in the binding complex that includes the membrane-associated protein and nuclease protein. The first and second dimerization domains specifically bind to each other or to a dimerization mediator, as desired. The terms “specific binding,” “specifically bind,” and the like, refer to the ability of different domains, e.g., first dimerization domain, second dimerization domain, dimerization mediator, to preferentially bind to each other relative to other molecules or moieties in a cell. In certain embodiments, the affinity between these binding pairs when they are specifically bound to each other in a binding complex is characterized by a K_{D} (dissociation constant) of 10⁻⁵ M or less, 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less, 10⁻¹³ M or less, 10⁻¹⁴ M or less, or 10⁻¹⁶ M or less (it is noted that these values can apply to other specific binding pair interactions mentioned elsewhere in this description, in certain embodiments).

As mentioned above, the first and second dimerization domains are domains that are capable of binding each other in a multimeric, e.g., dimeric, complex. As such, any two convenient polypeptide domains that are capable of forming a complex with each other may be selected for use as the first and second dimerization domains. The first and second dimerization domains may be included as part of the membrane-associated protein and the one or more nuclease proteins, respectfully, using any convenient method. In some cases, the membrane-associated protein and/or the one or more nuclease proteins are fusion proteins that have been engineered to include a dimerization domain. When present in a fusion protein, the dimerization domain may be separated from the membrane-associated protein and/or the nuclease protein by a linking sequence. In other cases, the dimerization domain may be a natural domain contained within the membrane-associated protein and/or the one or more nuclease proteins.

Any convenient set of dimerization domains may be employed. The first and second dimerization domains may be homodimeric, such that they are made up of the same sequence of amino acids, or heterodimeric, such that they are made up of differing sequences of amino acids. Dimerization domains may vary, where domains of interest include, but are not limited to: ligands of target biomolecules, such as ligands that specifically bind to particular proteins of interest (e.g., protein:protein interaction domains), such as SH2 domains, Paz domains, RING domains, transcriptional activator domains, DNA binding domains, enzyme catalytic domains, enzyme regulatory domains, enzyme subunits, domains for localization to a defined cellular location, recognition domains for the localization domain, the domains listed at: pawsonlab.mshri.on.ca/index.php?option=com_content&task=view&id=30&Itemid=63/, etc.

Dimerization domains of interest include, but are not limited to, protein domains of the iDimerize inducible homodimer (e.g., DmrB) and heterodimer systems (e.g., DmrA and DmrC) and the iDimerize reverse dimerization system (e.g., DmrD) (see e.g., Clontech.com Cat. Nos. 635068, 635058, 635059, 635060, 635069, 635088, 635090 and 635055) (See Clackson et al. (1998), Proc. Natl. Acad. Sci. USA 95(18): 10437-10442; Crabtree, G. R. & Schreiber, S. L. (1996), Trends Biochem. Sci. 21(11): 418-422; Jin et al. (2000), Nat. Genet. 26(1): 64-66; Castellano et al. (1999), Curr. Biol. 9(7): 351-360; Crabtree et al. (1997), Embo. J. 16(18): 5618-5628; Muthuswamy et al. (1999), Mol. Cell. Biol. 19(10): 6845-6857).

The first and second dimerization domains may be selected from DmrA and DmrC domains, DmrB domains, DmrD domains, dimerization domains of the dihydrofolate reductase system, dimerization domains of TAg and p53, and dimerization domains of SH2 and a PTRK protein.

Also of interest as dimerization domains are transcription activation domains. Transcription activation domains of interest include, but are not limited to: Group H nuclear receptor member transcription activation domains, steroid/thyroid hormone nuclear receptor transcription activation domains, synthetic or chimeric transcription activation domains, polyglutamine transcription activation domains, basic or acidic amino acid transcription activation domains, a VP16 transcription activation domain, a GAL4 transcription activation domains, an NF-κB transcription activation domain, a BP64 transcription activation domain, a B42 acidic transcription activation domain (B42AD), a p65 transcription activation domain (p65AD), or an analog, combination, or modification thereof.

As mentioned above, the first and second dimerization domains may also bind to a dimerization mediator to produce the desired complexes of membrane-associated protein and nuclease protein. In other words, a dimerization mediator may promote the complexation of the first and second dimerization domains, e.g., where both the first and second dimerization domains specifically bind to different regions of the dimerization mediator. Any convenient dimerization mediator may be employed. A dimerization mediator is a compound that induces proximity of the membrane associated and nuclease proteins under intracellular conditions. A dimerization mediator can be a homodimerizer or a heterodimerizer. By “induces proximity” is meant that two or more, such as three or more, including four or more, molecules are spatially associated with each other through a binding event mediated by the dimerization mediator compound. Spatial association is characterized by the presence of a binding complex that includes the dimerization mediator and the at least membrane associated and nuclease protein molecules. In the binding complex, each member or component is bound to at least one other member of the complex. In this binding complex, binding amongst the various components may vary. For example, the dimerization mediator may mediate a direct binding event between domains of membrane associated and nuclease protein molecules. The mediated binding event may be one that does not occur in the absence of the mediator, or one that occurs to a lesser extent in the absence of the mediator, such that the mediator results in enhanced dimer production as compared to control situations where the mediator is absent. For example, in the presence of the dimerization mediator, a first dimerization domain of a membrane associated protein may bind to a second dimerization domain of a nuclease protein molecule. The dimerization mediator may simultaneously bind to domains of the membrane associated and nuclease molecules, thereby producing the binding complex and desired spatial association. In some instances, the dimerization mediator induces proximity of the membrane associated and nuclease protein molecules, where these molecules bind directly to each other in the presence of the dimerization mediator.

Any convenient compound that functions as a dimerization mediator may be employed. A wide variety of compounds, including both naturally occurring and synthetic substances, can be used as dimerization mediators. Applicable and readily observable or measurable criteria for selecting a dimerization mediator include: (A) the ligand is physiologically acceptable (i.e., lacks undue toxicity towards the cell or animal for which it is to be used); (B) it has a reasonable therapeutic dosage range; (C) it can cross the cellular and other membranes, as necessary (where in some instances it may be able to mediate dimerization from outside of the cell), and (D) binds to the nuclease domains of the chimeric proteins for which it is designed with reasonable affinity for the desired application. A first desirable criterion is that the compound is relatively physiologically inert, but for its dimerization mediator activity. In some instances, the ligands will be non-peptide and non-nucleic acid.

Dimerization mediator compounds of interest include small molecules and are non-toxic. By small molecule is meant a molecule having a molecular weight of 5000 daltons or less, such as 2500 daltons or less, including 1000 daltons or less, e.g., 500 daltons or less. By non-toxic is meant that the inducers exhibit substantially no, if any, toxicity at concentrations of 1 g or more/kg body weight, such as 2.5 g or more/kg body weight, including 5 g or more/kg body weight. In one embodiment, the dimerization mediator is B/B homodimerizer or A/C heterodimerizer (Clontech).

One type of dimerization mediator of interest is a compound (as well as homo- and hetero-oligomers (e.g., dimers) thereof), that is capable of binding to an FKBP protein and/or to a cyclophilin protein. Such compounds include, but are not limited to: cyclosporin A, FK506, FK520, and rapamycin, and derivatives thereof. Many derivatives of such compounds are already known, including synthetic analogs of rapamycin, which can be adapted for use in the subject methods as desired.

In some embodiments, the dimerization mediator is a rapamycin analog (i.e., a rapalog). Any suitable rapalog may be modified for use as a dimerization mediator in the subject methods. As used herein, the term “rapalogs” refers to a class of compounds comprising the various analogs, homologs and derivatives of rapamycin and other compounds related structurally to rapamycin. Rapalogs include but are not limited to, variants of rapamycin having one or more of the following modifications relative to rapamycin: demethylation, elimination or replacement of the methoxy at C7, C42 and/or C29; elimination, derivatization or replacement of the hydroxy at C13, C43 and/or C28; reduction, elimination or derivatization of the ketone at C14, C24 and/or C30; replacement of the 6-membered pipecolate ring with a 5-membered prolyl ring; and elimination, derivatization or replacement of one or more substituents of the cyclohexyl ring or replacement of the cyclohexyl ring with a substituted or unsubstituted cyclopentyl ring. Rapalogs, as that term is used herein, do not include rapamycin itself, and in some instances do not contain an oxygen bridge between C1 and C30. Rapalogs that may be used as dimerization mediators in embodiments of the invention include, but are not limited to, those compounds described in: U.S. Pat. No. 7,067,526; and U.S. Pat. No. 7,196,192; the disclosures of which are herein incorporated by reference. Further illustrative examples of rapalogs are disclosed in the following documents: U.S. Pat. No. 6,693,189; U.S. Pat. No. 6,984,635, WO9641865, WO9710502, WO9418207, WO9304680, U.S. Pat. No. 5,527,907, U.S. Pat. No. 5,225,403, WO9641807, WO9410843, WO9214737, U.S. Pat. No. 5,484,799, U.S. Pat. No. 5,221,625, WO9635423, WO9409010, WO9205179, U.S. Pat. No. 5,457,194, U.S. Pat. No. 5,210,030, WO9603430, WO9404540, U.S. Pat. No. 5,604,234, U.S. Pat. No. 5,457,182, U.S. Pat. No. 5,208,241, WO9600282, WO9402485, U.S. Pat. No. 5,597,715, U.S. Pat. No. 5,362,735, U.S. Pat. No. 5,200,411, WO9516691, WO9402137, U.S. Pat. No. 5,583,139, U.S. Pat. No. 5,324,644, U.S. Pat. No. 5,198,421, WO9515328, WO9402136, U.S. Pat. No. 5,563,172, U.S. Pat. No. 5,318,895, U.S. Pat. No. 5,147,877, WO9507468, WO9325533, U.S. Pat. No. 5,561,228, U.S. Pat. No. 5,310,903, U.S. Pat. No. 5,140,018, WO9504738, WO9318043, U.S. Pat. No. 5,561,137, U.S. Pat. No. 5,310,901, U.S. Pat. No. 5,116,756, WO9504060, WO9313663, U.S. Pat. No. 5,541,193, U.S. Pat. No. 5,258,389, U.S. Pat. No. 5,109,112, WO9425022, WO9311130, U.S. Pat. No. 5,541,189, U.S. Pat. No. 5,252,732, U.S. Pat. No. 5,093,338, WO9421644, WO9310122, U.S. Pat. No. 5,534,632, U.S. Pat. No. 5,247,076, and U.S. Pat. No. 5,091,389, the disclosures of which are herein incorporated by reference.

Dimerization domains that may be incorporated into the membrane-associated and nuclease proteins for use with such dimerization mediators may vary. In some instances, the dimerization domains may be selected from naturally occurring peptidyl-prolyl isomerase family proteins or derivatives, e.g., mutants (including point and deletion), thereof. Examples of domains of interest for these embodiments include, but are not limited to: FKBP, FRB, and the like.

FKBP dimerization domains may contain all or part of the peptide sequence of an FKBP domain. Of interest are those domains that are capable of binding to a corresponding dimerization mediator, e.g., a rapalog, with a Kd value of, e.g., 100 nM or less, such as about 10 nM or less, or even about 1 nM or less, as measured by direct binding measurement (e.g. fluorescence quenching), competition binding measurement (e.g. versus FK506), inhibition of FKBP enzyme activity (rotamase), or other assay methodology. The peptide sequence of a FKBP domain of interest may be modified to adjust the binding specificity of the domain for a dimerization mediator, e.g., by replacement, insertion or deletion of 25 or less, such as 20 or less, 15 or less, 10 or less, such as 5 or less, or 3 or less amino acid residues. A FRB domain of interest includes domains capable of binding to a complex of an FKBP protein and dimerization mediator, e.g., rapalog. The FRB fusion protein may bind to that complex with a Kd value of about 200 μM or less, such as about 10 μM or less, 2 μM or less, or even 1 μM or less, as measured by conventional methods. The FRB domain is of sufficient length and composition to maintain high affinity for a complex of the rapalog with the FKBP fusion protein. For example, myristolated FKBP in conjunction with a rapamycin analog can translocate a FRB fusion protein to a plasma membrane. (Nat. Methods., 6:415-418, 2005)

Another type of dimerization mediator compound of interest is an alkenyl substituted cycloaliphatic (ASC) dimerization mediator compound. ASC dimerization mediator compounds include a cycloaliphatic ring substituted with an alkenyl group. In certain embodiments, the cycloaliphatic ring is further substituted with a hydroxyl and/or oxo group. In some cases, the carbon of the cycloaliphatic ring that is substituted with the alkenyl group is further substituted with a hydroxyl group. The cycloaliphatic ring system may be an analog of a cyclohex-2-enone ring system. In some embodiments, the ASC dimerization mediator compound includes a cyclohexene or a cyclohexane ring, such as is found in a cyclohexenone group (e.g. a cyclohex-2-enone), a cyclohexanone group, a hydroxy-cyclohexane group, a hydroxy-cyclohexene group (e.g., a cyclohex-2-enol group) or a methylenecyclohexane group (e.g. a 3-methylenecyclohexan-1-ol group); where the cycloaliphatic ring is substituted with an alkenyl group of about 2 to 20 carbons in length, that includes 1, 2, 3, 4, 5, 6, 7, 8, 9 or 10 unsaturated bonds. In certain embodiments, the alkenyl substituent includes a conjugated series of unsaturated bonds. In particular embodiments, the alkenyl substituent may be 4 carbons in length and include 2 conjugated double bonds. In another embodiment, the alkenyl substituent is conjugated to the cycloaliphatic ring system. Further details of such compounds are disclosed in WO/2011/163029; the disclosure of which is herein incorporated by reference.

Where the dimerization domain is an ASC inducer compound, such as abscisic acid, ASC binding dimerization domains of interest include, but are not limited to: the abscisic acid binding domains of the pyrabactin resistance (PYR)/PYR1-like (PYL)/regulatory component of ABA receptor (ROAR) family of intracellular proteins. The PYR/PYL/ROAR abscisic acid binding domains are those domains or regions of PYR/PYL/ROAR proteins, (e.g., pyrabactin resistance 1, PYR1-Like proteins, etc.) that specifically bind to abscisic acid. Accordingly, ASC inducer binding domains include a full length PYR1 or PYL protein (e.g., PYL1, PYL 2, PYL 3, PYL 4, PYL 5, PYL 6, PYL, PYL 8, PYL 9, PYL 10, PYL11, PYL12, PYL13), as well as portions or mutants thereof that bind to abscisic acid, e.g., amino acid residues 33-209 of PYL1 from Arabidopsis thaliana. Additional examples of suitable ASC binding dimerization domains include PP2C inducer domains. The PP2C inducer domains are those PYR/PYL binding domains found in group A type 2 C protein phosphatases (PP2Cs), where PP2Cs have PYL(+ABA) binding domains. Accordingly, ASC inducer binding domains include the full length PP2C proteins (e.g., ABI1), as well as portions or mutants thereof that bind to abscisic acid, e.g., amino acid residues 126-423 of ABI1 from Arabidopsis thaliana. In some instances, the PP2C ASC inducer domain is a phosphatase negative mutant, e.g., a mutant of PP2C that retains its ability to specifically bind to PYR/PYL (+ABA) and yet has reduced if not absent phosphatase activity.

Another type of dimerization mediator compound of interest is an N-oxalyl-pipecolyl or N-oxalyl-prolyl-type compound. N-oxalyl-pipecolyl and N-oxalyl-prolyl-type compounds include immunophilin multimerizing agents described in WO 1996/06097, the disclosure of which is herein incorporated by reference.

Another type of dimerization mediator compound of interest is an oligonucleotide ligand containing compound. Oligonucleotide ligand containing compounds include multi-functional oligonucleotide ligands described in WO 1993/03052, the disclosure of which is herein incorporated by reference.

In some instances, the dimerization mediator is a modifiable dimerization mediator. In some instances, the dimerization mediator is modifiable (e.g., a MDM). A MDM is a compound that reversibly induces proximity of the membrane-associated protein and the nuclease protein in a sample under suitable conditions, where proximity may be reversed by the application of a stimulus. Application of the stimulus to the sample modifies a modifiable group of the MDM, thereby changing the nature of the MDM such that the modified MDM is no longer capable of inducing or maintaining proximity of the membrane-associated protein and the nuclease protein.

By “reversibly induces proximity” or “reverse the induction of proximity” is meant that the spatial association of membrane-associated protein and the nuclease protein, mediated by a MDM, may be reversed upon application of a suitable stimulus (e.g., a photon, a chemical agent or an enzyme) that modifies the MDM. Application of a suitable stimulus results in dissociation of the membrane-associated protein and the nuclease protein components of the dimeric complex. In some cases, the stimulus may be described as a modifying stimulus, e.g., a stimulus that results in modification of the modifiable group. In certain embodiments, application of a stimulus is not application of a competitive inhibitor of binding of the MDM to domains of the membrane-associated protein and nuclease protein. In certain embodiments, application of a stimulus is not dilution of the sample.

Application of a suitable stimulus to the sample will modify the modifiable group to result in modification of the MDM, e.g., a change in the nature of the MDM molecule that alters its binding properties. In some embodiments, the modified MDM has significantly reduced affinity for the dimerization domains of the membrane-associated protein and/or the nuclease protein, e.g., an affinity that is reduced by 2-fold or more, such as 3-fold or more, 4-fold or more, 5-fold or more, 10-fold or more, 30-fold or more, 50-fold or more, 100-fold or more, or even 1000-fold or more, as compared to the corresponding affinity of the unmodified MDM. In some embodiments, the Kd value of a MDM (e.g., a rapalog-derived or a ASC-derived MCIP), for a dimerization domain (e.g., a FKBP domain or a ASC binding domain) may be raised from about 10 nM or less (e.g., about 3 nM or less or about 1 nM or less) to about 20 nM or more, such as about 30 nM or more, about 40 nM or more, about 50 nM or more, about 100 nM or more, about 200 nM or more, about 500 nM or more, or even about 1 μM or more.

In some embodiments, the MDM includes a cleavable group where application of the stimulus cleaves the cleavable group. Application of the stimulus may produce two cleaved MDM products, where each product independently retains affinity for only one of the first and second dimerization domains. In some embodiments, the MDM includes a cleavable linker connecting a first binding moiety that specifically binds the first dimerization domain, and a second binding moiety that specifically binds the second dimerization domain, such that cleavage of the linker leads to dissociation of the membrane-associated protein and the nuclease protein. In other embodiments, application of the stimulus produces a modified MDM where one of the first and second binding moieties is changed in nature such that it has significantly reduced affinity (e.g., as described herein) for a corresponding dimerization domain. In such cases, the binding affinity of the other binding moiety may be unaffected, or alternatively, it may also be significantly reduced (e.g., as described herein).

The MDM may include a first binding moiety (A) that specifically binds to a first dimerization domain and a second binding moiety (B) that specifically binds to a second dimerization domain, and a modifiable group (X). X may be a part of A or a part of B, or X may be connected to A and/or B via a linker. In some instances, the MDM specifically binds the first and second dimerization domains independently, e.g., formation of a ternary complex may occur via initial binding of the MDM to either the first or the second dimerization domain. In other instances, specific binding of the MDM to the second dimerization domain is dependent on prior formation of a MDM/first dimerization domain complex. In this context, by “dependent” is meant that the second nuclease molecule has a higher affinity for the complex of MDM/first dimerization domain than it has for the MDM alone. In some embodiments, MDMs which form such ternary complexes include a first binding moiety (A) that specifically binds a first dimerization domain (e.g., a rapalog that specifically binds a FKBP domain, or an alkenyl substituted cycloaliphatic (ASC) inducer compound that specifically binds an PYL ASC binding domain), and the second binding moiety (B) that specifically binds a second dimerization domain (e.g., a rapalog that specifically binds a FRB domain, or an ASC inducer compound that specifically binds an ABI ASC binding domain) where binding of the second dimerization domain is dependent on the prior binding of A and the first dimerization domain. In certain cases, the complex of MDM/first dimerization domain specifically binds the second dimerization domain without direct contacts being formed between the MDM and the nuclease protein. In such cases, the MDM mediates the binding of the membrane-associated protein and the nuclease protein.

The modifiable group (X) may be included at any convenient position in the structure of an MDM. In some cases, the modifiable group (X) is part of the first binding moiety (A) or is part of the second binding moiety (B). In some cases, X may be included in that part of the structure which specifically binds the first dimerization domain (e.g., a FKBP domain or ASC binding domain), or alternatively, may be included in that part of the structure which specifically binds the second dimerization domain (e.g., a FRB domain or a ASC binding domain). In other cases, X may be separate from the binding moieties A and B. As such, X may be located at a position of the structure that is not involved in specific binding interactions with the first or second dimerization domains, e.g., in a linker that connects A and B.

Of interest as MDMs are the MCIPs and dimerization systems described in US Application Publication No. 2014/0080137, which is herein incorporated by reference.

### Microvesicle Inducer

As used herein, “microvesicle inducer” refers to an agent that promotes (i.e., enhances) the production of microvesicles from a cell. In some cases, the microvesicle inducer is a molecule that does not become part of the microvesicles, but where the presence of the microvesicle inducer results in the cell producing microvesicles. In other cases, the microvesicle inducer becomes part of the produced microvesicles. In some instances, the production of microvesicles can be accomplished through the overexpression of a microvesicle inducer within a mammalian cell. In certain cases, overexpression of a microvesicle inducer in a cell results in shedding of microvesicles into the medium surrounding the transfected cell.

The microvesicle inducer may be a protein, small molecule inducer, endogenous “cell-blebbing” e.g., during apoptosis, and the like. Protein microvesicle inducers include, but are not limited to: proteins that induce membrane budding, viral membrane fusion proteins, small molecule inducers of vesicle formation, etc.

In some cases, the microvesicle inducer is a protein that induces membrane budding such that the production of microvesicles is enhanced. As used herein, the expression “protein which induces membrane budding” refers to any protein that can promote the deformation of lipid bilayers and mediate the formation of vesicles. Any convenient cellular or viral proteins may be utilized to induce membrane budding. Cellular proteins of interest that induce membrane budding include, but are not limited to, proteolipid protein PLP1 (Trajkovic et al. 2008 Science, vol 319, p 1244-1247), clathrin adaptor complex AP1 (Camus et al., 2007. Mol Biol Cell vol 18, p3193-3203), proteins modifying lipid properties such as fleippase, scramblase, proteins which facilitate secretion via a non-classical pathway such as TSAP6 (Yu et al. 2006 Cancer Res vol 66, p4795-4801) and CHMP4C (Yu et al. 2009, FEBS J. vol 276, p2201-2212). Viral proteins of interest that induce membrane budding include, but are not limited to, tetherin/CD317 antagonists such as the Vpu protein of HIV (Neil et al. 2008. Nature vol 451, p425-4431) and various viral structural proteins such as retroviral GAG (Camus et al., 2007. Mol Biol Cell vol 18, p3193-3203) and Ebola VP40 (Timmins et al., Virology 2001).

In some cases, the microvesicle inducer is a viral membrane fusion protein (e.g., viral fusion glycoprotein). The viral membrane fusion protein may be a class I viral membrane fusion protein such as the influenza-virus hemagglutinin, a class II viral membrane fusion protein or a class III viral membrane fusion protein (e.g., as described in Backovic et al., Curr. Opin. Struct. Biol. 2009, 19(2): 189-96; Courtney et al., Virology Journal 2008, 5: 28). In some embodiments, the viral membrane fusion protein is a class I viral membrane fusion protein. Class I viral membrane fusion proteins of interest include, but are not limited to, Baculovirus F proteins, F proteins of the nucleopolyhedrovirus (NPV) genera, such as Spodoptera exigua MNPV (SeMNPV) F protein and Lymantria dispar MNPV (LdMNPV) F protein. The microvesicle inducer may be a class III viral membrane fusion protein, where Class III viral membrane fusion proteins of interest include, but are not limited to, rhabdovirus G (such as the fusogenic protein G of the Vesicular Stomatatis Virus (VSV-G)), herpesvirus gB (such as the glycoprotein B of Herpes Simplex virus 1 (HSV-1 gB)), EBV gB, thogotovirus G, baculovirus gp64 (such as Autographa California multiple NPV (AcMNPV) gp64), and the Borna disease virus (BDV) glycoprotein (BDV G). In certain instances, the viral membrane fusion protein is VSV-G or baculovirus gp64.

In certain embodiments, the microvesicle inducer is VSV-G, such as the VSV-G polypeptide as defined in GenBank AN: M35219.1, or any functional fragments or their functional derivatives retaining fusogenic properties. As used herein with respect to viral membrane fusion proteins, the term “fusogenic” refers to a viral protein that can induce the fusion of the membrane of the microvesicles to the plasma membrane of the target cell. VSV-G fusogenic polypeptides of interest include but are not limited to, those described in U.S. Pat. Nos. 7,323,337; 5,670,354; 5,512,421; 20100167377; and the like.

Also of interest are small molecule inducers of vesicle formation. Small molecule inducers of vesicle formation include, but are not limited to: Apoptosis inducer causing cell blebbing e.g. Staurosporin, and the like.

Where desired, a microvesicle inducer may be provided in the cell using any convenient protocol. For example, the cell may be configured to express the microvesicle inducer from a coding sequence in the cell, or a microvesicle inducer may be added to the cells using any convenient method. Methods of adding a microvesicle inducer to a cell include, but are not limited to, transfection or transduction of the cell with a construct encoding a microvesicle inducing protein, and contact of the cell with a chemical inducer (e.g., a small molecule), etc.

### Microvesicle Producing Cells

The present invention provides a method of preparing a microvesicle containing a nuclease. The method comprises: (a) maintaining a packaging cell comprising: (i) a membrane-associated protein comprising at least one first dimerization domain, and (ii) a nuclease comprising at least one second dimerization domain, and (b) producing a microvesicle that contains the nuclease from the packaging cell under sufficient conditions. In a preferred embodiment, the nuclease is selected from the group consisting of a Cas protein, and Argonaute nuclease.

Any convenient cell capable of producing microvesicles may be utilized. In some instances, the cell is a eukaryotic cell. Cells of interest include eukaryotic cells, e.g., animal cells, where specific types of animal cells include, but are not limited to: insect, worm, avian or mammalian cells. Various mammalian cells may be used, including, by way of example, equine, bovine, ovine, canine, feline, murine, non-human primate and human cells. Among the various species, various types of cells may be used, such as hematopoietic, neural, glial, mesenchymal, cutaneous, mucosal, stromal, muscle (including smooth muscle cells), spleen, reticulo-endothelial, epithelial, endothelial, hepatic, kidney, gastrointestinal, pulmonary, fibroblast, and other cell types. Hematopoietic cells of interest include any of the nucleated cells which may be involved with the erythroid, lymphoid or myelomonocytic lineages, as well as myoblasts and fibroblasts. Also of interest are stem and progenitor cells, such as hematopoietic, neural, stromal, muscle, hepatic, pulmonary, gastrointestinal and mesenchymal stem cells, such as ES cells, epi-ES cells and induced pluripotent stem cells (iPS cells). Specific cells of interest include, but are not limited to: mammalian cells, e.g., HEK-293 and HEK-293T cells, COS7 cells, Hela cells, HT1080, 3T3 cells etc.; insect cells, e.g., High5 cells, Sf9 cells, Sf21 and the like. Additional cells of interest include, but are not limited to, those described in US Publication No. 20120322147, the disclosure of which cells are herein incorporated by reference.

As summarized above, the cells are ones that include a membrane-associated protein, a nuclease protein and, where desired, a microvesicle inducer, e.g., as described in greater detail above. As such, the cells are cells that have been engineered to include the membrane-associated and nuclease proteins. The cells may comprise a first expression cassette comprising a first coding sequence for encoding the membrane-associated protein comprising a first dimerization domain; and a second expression cassette comprising a second coding sequence for encoding the nuclease comprising a second dimerization domain.

The protocol by which the cells are engineered to include the desired proteins may vary depending on one or more different considerations, such as the nature of the target cell, the nature of the molecules, etc. The cell may include expression constructs having coding sequences for the proteins under the control of a suitable promoter, where the promoter may be an inducible promoter or constitutively active. The coding sequences will vary depending on the particular nature of the protein encoded thereby, and will include at least a first domain that encodes the dimerization domains and a second domain that encodes the membrane associated or nuclease domains. The two domains may be joined directly or linked to each other by a linking domain. The domains encoding these fusion proteins are in operational combination, i.e., operably linked, with requisite transcriptional mediation or regulatory element(s). Requisite transcriptional mediation elements that may be present in the expression module include promoters (including tissue specific promoters), enhancers, termination and polyadenylation signal elements, splicing signal elements, and the like. Of interest in some instances are inducible expression systems. The various expression constructs in the cell may be chromosomally integrated or maintained episomally, as desired. Accordingly, in some instances the expression constructs are chromosomally integrated in a cell. Alternatively, one or more of the expression constructs may be episomally maintained, as desired. The expression constructs may be expressed stably in the cell or transiently, as needed/desired.

The cells may be prepared using any convenient protocol, where the protocol may vary depending on nature of the cell, the location of the cell, e.g., in vitro or in vivo, etc. Where desired, vectors, such as plasmids or viral vectors, may be employed to engineer the cell to express the various system components, e.g., membrane-associated and nuclease proteins, optional microvesicle inducer, etc., as desired. Protocols of interest include those described in published PCT application WO1999/041258, the disclosure of which protocols are herein incorporated by reference.

Depending on the nature of the cell and/or expression construct, protocols of interest may include electroporation, particle gun technology, calcium phosphate precipitation, direct microinjection, viral infection and the like. The choice of method is generally dependent on the type of cell being transformed and the circumstances under which the transformation is taking place (i.e., in vitro, ex vivo, or in vivo). A general discussion of these methods can be found in Ausubel, et al, Short Protocols in Molecular Biology, 3rd ed., Wiley & Sons, 1995. In some embodiments, lipofectamine and calcium mediated gene transfer technologies are used. After the subject nucleic acids have been introduced into a cell, the cell may be incubated, normally at 37° C., sometimes under selection, for a period of about 1-24 hours in order to allow for the expression of the chimeric protein. In mammalian target cells, a number of viral-based expression systems may be utilized to express a chimeric protein(s). In cases where an adenovirus is used as an expression vector, the chimeric protein coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the chimeric protein in infected hosts. (e.g., see Logan & Shenk, Proc. Natl. Acad. Sci. USA 81:355-359 (1984)). The efficiency of expression may be enhanced by the inclusion of appropriate transcription enhancer elements, transcription terminators, etc. (see Bittner et al., Methods in Enzymol. 153:51-544 (1987)).

Where long-term, high-yield production of the chimeric proteins is desired stable expression protocols may be used. For example, cell lines, which stably express the chimeric protein, may be engineered. Rather than using expression vectors which contain viral origins of replication, host cells can be transformed with chimeric protein expression cassettes and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into a chromosome and grow to form foci which in turn can be cloned and expanded into cell lines. In addition, the coding sequences can be inserted by means of zinc finger nucleases, meganucleases, TAL effector nucleases, or RGEN mediated methods followed by HR or homologous recombination into “safe harbor” regions of the human or other genomes. Safe harbor regions of interest include regions that are single copy, diploid or aneuploid and are not near genes that regulate growth or are likely to cause cancerous transformation or other non-therapeutic perturbations if not Properly Regulated.

Production of Microvesicles from Microvesicle Producing Cells

Aspects of the methods include maintaining a microvesicle producing cell, e.g., as described above, under conditions sufficient to produce one or more microvesicles from the cell, where the microvesicles include a dimerization complex of a membrane associated protein and a nuclease protein. Any convenient methods of maintaining the cell under conditions sufficient to produce a microvesicle may be utilized in the subject methods. In some cases, the cell is maintained for a period of time ranging from 30 minutes to 1 week, such as from 1 hour to 3 days, 1 hour to 2 days, including 1 hour to 24 hours. In some instances, the cell is maintained for a period of time ranging from 30 minutes to 72 hours, such as 2 to 72, 6 to 72, 12 to 72, 24 to 72, 36 to 72, and 48 to 72 hours. The cell may be maintained at a temperature that supports microvesicle production from the cell, where temperatures of interest include 4 to 42° C., such as 15 to 37° C. The cell may be maintained in a suitable culture medium, as desired, where culture media of interest include, but are not limited to: DMEM, HAMs F12, RPMI1640, serum free conditions, and the like.

Depending on the nature of the cell, aspects of the methods may include a step of inducing expression of the one or more of the membrane associated protein, the nuclease protein and the microvesicle inducer, if present. Expression of one or more of these proteins may be controlled by an inducible promoter, such as an inducible promoter of a Lac-based system or a Tet-based system. In such instances, the methods may include introducing into the cell an expression inducer, e.g., by contacting the cell with a medium that includes the inducer, etc.

Where desired, aspects of the methods may include contacting a cell with a microvesicle inducer in a manner sufficient to cause microvesicle production from the cell. For example, where the microvesicle inducer is a small molecule inducer, the methods may include contacting the cell with a medium that includes a sufficient concentration of the microvesicle inducer. In some instances, microvesicle production may be induced by modifying the cell culture conditions of the cell, such as modifying the temperature, e.g., to a value ranging from 15 to 42° C., modifying the Ca²⁺ concentration, e.g., as described in Biochemistry. 1998 Nov. 3; 37(44):15383-91, etc.

Depending on the nature of the cell, aspects of the methods may include a step of introducing into the cell a dimerization mediator. The dimerization mediator may be introduced into the cell using any convenient protocol. The particular protocol that is employed may vary, e.g., depending on whether the microvesicle producing cell is in vitro or in vivo. For in vitro protocols, introduction of the dimerization mediator into the cell may be achieved using any convenient protocol. In some instances, the sample includes cells that are maintained in a suitable culture medium, and the dimerization mediator is introduced into the culture medium. For in vivo protocols, any convenient administration protocol may be employed. Depending upon the binding affinity of the dimerization mediator, the response desired, the manner of administration, e.g., i.v., s.c., i.p., oral, etc., the half-life, the number of cells present, various protocols may be employed.

In some embodiments, the method further includes separating the microvesicle from the cell. Any convenient separation protocol may be employed. Examples of suitable separation protocols include, but are not limited to: filtration, centrifugation, precipitation, surface and/or antibody based capture and the like. In some cases, microvesicles are harvested from the cell supernatant post-transfection. In certain cases, the microvesicles may be isolated by ultracentrifugation, e.g., at 110,000 g for 1.5 hours, or by centrifugation, e.g., at 7500 g for 16 hours. In certain instances, the microvesicles may be frozen and stored at −80° C. without losing their ability to transfer material to the target cell. In some embodiments, the microvesicles do not include any nucleic acid coding for the nuclease protein of interest. In certain embodiments, the microvesicles are virus free.

The amount of the nuclease protein in the microvesicle may be evaluated at any convenient time during the subject methods, e.g., during the maintaining of cells step, or before or after an optional separating step, and utilizing any convenient method.

In certain embodiments, a given method only employs a single membrane associated protein/nuclease protein pair. In yet other embodiments, given method may employ two or more distinct membrane associated/nuclease protein pairs, such as where the production of microvesicles having two or more nuclease proteins packaged therein is desired. In yet other embodiments, two or more distinct nuclease proteins may be configured to dimerize to a common dimerization domain of membrane associated protein, such that one has a single membrane associated protein with two or more distinct nuclease proteins.

### Protein Enriched Microvesicles

Aspects of the invention further include protein-enriched microvesicles, e.g., such as produced by the methods described above. The microvesicles may include one or more membrane-associated proteins and one or more nuclease proteins, e.g., as described above, inside of a lipid bilayer envelope. As the microvesicles are produced from microvesicle producing cells, e.g., as described above, the lipid bilayer component of the microvesicle includes membrane components of the cell from which the microvesicle is produce, e.g., phospholipids, membrane proteins, etc. In addition, the microvesicle has a cytosol that includes components found in the cell from which the microvesicle is produced, e.g., solutes, proteins, nucleic acids, etc., but not all of the components of a cell, e.g., they lack a nucleus. In some embodiments, the microvesicles are considered to be exosome-like. The microvesicles may vary in size, and in some instances have a diameter ranging from 30 and 300 nm, such as from 30 and 150 nm, and including from 40 to 100 nm.

In some cases, in the microvesicle, the membrane-associated protein and the nuclease protein are present in a dimerized complex. In some instances, the first and second dimerization domains are specifically bound to each other in a dimerized complex. In other instances, the first and second dimerization domains are bound to each other by a dimerization mediator, e.g., as described above. In some instances, the microvesicle includes a microvesicle inducer, e.g., a viral membrane fusion protein, such as VSV-G. In some embodiments, the first dimerization domain of the membrane-associated protein contacts the cytosol of the microvesicle.

A given microvesicle may be enriched with a single nuclease protein or two or more distinct nuclease proteins. Where two or more distinct nuclease proteins are present in the microvesicle, each of the nuclease proteins may dimerize with the same membrane-associated protein or each nuclease protein may dimerize with its own membrane-associated protein, as desired. As such, in some instances a microvesicle may include a population of dimerized complexes that have a common membrane-associate protein but two or more distinct nuclease proteins represented in the population. In other instances, the population of dimerized complexes may include two or more distinct nuclease proteins each dimerized with a distinct membrane-associated protein. As such, the microvesicle may further include a second nuclease protein comprising a third dimerization domain.

Microvesicle Mediated Protein Delivery into a Cell

As summarized above, aspects of the invention include methods of introducing a protein into a target cell. Such methods include contacting the target cell with a microvesicle, e.g., as described above, where the microvesicle may be present in a composition of a population of microvesicles (for example where the number of microvesicles ranges from 10³ to 10¹⁶, such as 10⁴ to 10¹³, including as 10⁴ to 10⁹), under conditions sufficient for the microvesicle to fuse with the target cell and deliver the nuclease protein contained in the microvesicle into the cell. Any convenient protocol for contacting the cell with the microvesicle may be employed. The particular protocol that is employed may vary, e.g., depending on whether the target cell is in vitro or in vivo. For in vitro protocols, target cells may be maintained with microvesicles in a suitable culture medium under conditions sufficient for the microvesicles to fuse with the target cells. Examples of suitable culture media include, but are not limited to: DMEM, Hams F12, RPMI1640 and the like. The target cells and microvesicles may be maintained for a period of time sufficient for the microvesicles to fuse with the cells, where the period of time ranges, in some instances, from 5 mins to 72 hrs, such as 30 mins to 2 hrs. The target cells and microvesicles may be maintained at a suitable temperature, e.g., a temperature ranging from 4° C. to 42° C., such as 15° C. to 37° C. For in vivo protocols, any convenient administration protocol may be employed. Depending upon the tropism of the microvesicle and the target cell, the response desired, the manner of administration, e.g. i.v., s.c., i.p., oral, etc., the half-life, the number of cells present, various protocols may be employed.

In some embodiments, the method further includes disrupting (i.e., dissociating) the dimerized or multierized complex in the microvesicle. Dissociation of the dimerized or multierized complex may lead to faster release of the nuclease protein in the cell resulting in a faster and or larger biological response in the cell. The dimerized or multimerized complex may be disrupted using any convenient protocol. In one embodiment, the dimerization domains can be dissociated by reducing the concentration of a dimerization mediator. Typically microvesicles are formed in packaging cells in the presence of a dimerization mediator in the culture medium. When microvesicles are introduced to target cells that are cultured in the absence of the dimerization mediator, the concentration of the dimerization mediator decreases, thus causing dissociation of the dimerized or multimerized complex.

In some embodiments, the method may include contacting the microvesicle with a solubilizer, i.e., dimerization disruptor, compound to dissociate the complex of the nuclease protein and the membrane-associated protein in the microvesicle. Any convenient solubilizer compounds may be utilized. Solubilizer compounds of interest include, but are not limited to, the D/D solubilizer compound (Clontech, Mountain View, Calif.). The D/D solubilizer may dissociate dimeric complexes that include DmrD dimerization domains, by binding to the DmrD domain in a manner that disrupts (reverses) their self-association. The D/D solubilizer also dissociates complexes that include the DmrB homodimerization domain.

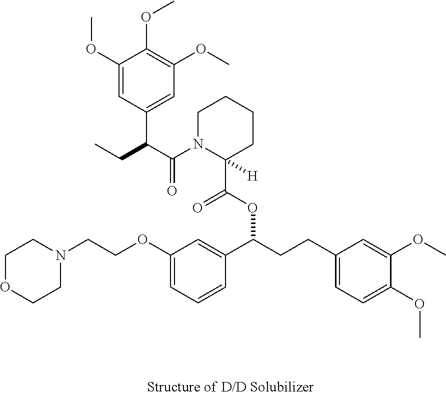

In those instances where the dimerization complex includes a dimerization mediator, excess mediator may be introduced into the microvesicles, e.g., as described above, in order to dissociate the complex. Where the dimerization mediator is a modifiable dimerization mediator, dissociation of the complex may include applying a stimulus to the microvesicle to modify a modifiable dimerization mediator to dissociate the dimeric complex of nuclease protein and membrane-associated protein. In such embodiments, the target cells may be maintained for any convenient period of time prior to application of a stimulus, such as a photon, a chemical agent or an enzyme, to the cells. As such, further aspects of embodiments of the method include application of a stimulus to a sample that includes the target cells and the microvesicles to modify the modifiable dimerization domain and disrupt dimerization complex of the membrane-associated protein and the nuclease protein, respectively.

In certain instances, the method further includes assessing, i.e., evaluating, a function of the nuclease protein in the cell. Once the nuclease protein of interest has been introduced into a target cell, the occurrence of a particular biological event triggered by the introduction of the nuclease protein into the cell may be evaluated. Evaluation of the cells may be performed using any convenient method, and at any convenient time before, during and/or after contact of the microvesicles with the cells. Evaluation of the cells may be performed continuously, or by sampling at one or more time points during the subject method. In some embodiments, the evaluating step is performed prior to the contacting step. In certain embodiments, the evaluating step is performed prior to application of a stimulus. In certain cases, the evaluation is performed using a cell-based assay that measures the occurrence of a biological event triggered by the nuclease protein. Any observable biological property of interest may be used in the evaluating steps of the subject methods.

Embodiments of the methods are characterized by providing for transient activity of the delivered protein in the target cell. By transient is meant that the delivered protein remains active for a limited period of time, and in some embodiments the limited period of time ranges from 10 min to 96 hr, including 2 hr to 48 hr. It yet other embodiments, the protein may remain active for longer period of time, e.g., 96 hr or longer, such as 100 hr or longer, including 200 hr or longer.

In a second aspect, the present invention is directed to a microvesicle comprising: (i) a membrane-associated protein comprising at least one first dimerization domain, (ii) a carrier protein comprising at least one second dimerization domain, and (iii) a solute that binds to the carrier protein. The solute may be any molecule that is capable to bind to a carrier protein. In general, the solute may be DNA, RNA, protein, lipid, carbohydrate, an organelle such as ribosomes, mitochondria, and small molecules under molecular weight of 5000 daltons.

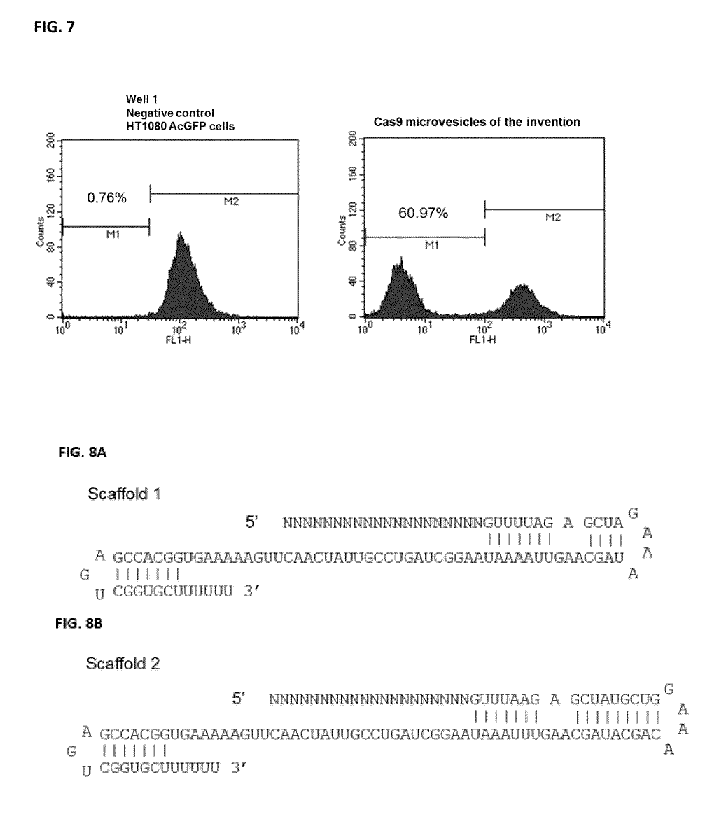

In one embodiment, the solute is a single guide RNA and the carrier protein is a nuclease. Necleases have been described above in details. For example, the nuclease may be CAS nuclease or Argonaute nuclease. In a preferred embodiment, the nuclease is CAS9, CAS9 mutant without nuclease activity (e.g., Cas9 D10A, H840A double mutant), or Nickase mutant of Cas9 (e.g. Cas9 D10A mutant or Cas9 H840A). When the nuclease is CAS 9 or its mutant, the single guide RNA for example, may comprise the scaffold nucleic acid sequence of SEQ ID NO: 1 or 2 as shown in FIGS. 8A and 8B.

In one embodiment, the solute is RNA and the carrier protein is a RNA binding protein having one or more RNA binding domains. The RNA binding proteins including, but not limited to, bacteriophage MS2 coat protein (known to the person skilled in the art as MS2 binding protein/domain), lambdaN22, PUMILIO1, and SRSF1 deletion mutants.

MS2 is a bacteriophage coat protein that specifically binds to the stem-loop structure of a particular viral RNA sequence. It has been shown, that any RNA, containing this stem-loop structure, will be bound by the MS2-binding domain, independent which RNA sequence it is part of. Fusion proteins of GFP and the MS2-binding domain have been used for live cell imaging to locate an exogenous mRNA of interest containing repeats of the stem-loop structure; the GFP-MS2 fusion protein binds to the mRNA via the repeat stem-loop domains (Tyagi S., Nature Methods 6: 331-338 (2009)). This application of the MS2-binding-domain underlines its ability to specifically bind to RNA sequence containing this specific stem loop sequence in live cells.

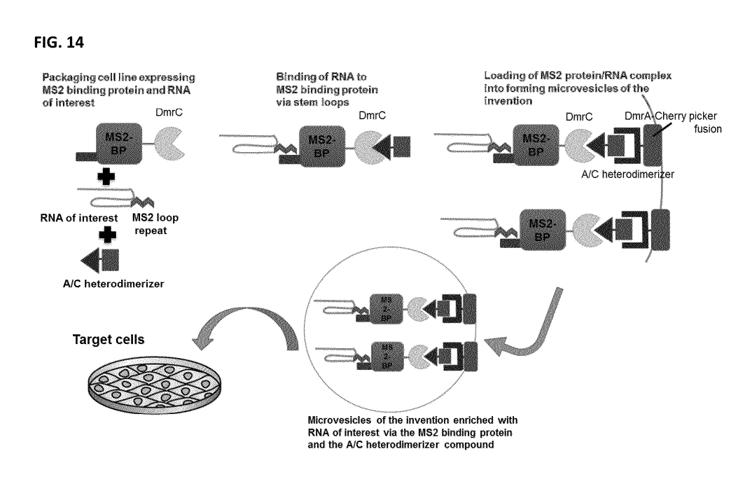

The present invention provides using the MS2-binding-domain to deliver a RNA of interest via microvesicles into target cells. As illustrated in FIG. 14, the mRNA of interest containing the stem-loop sequence, which may be introduced into the 3′untranslated region of a target mRNA, is co-expressed with the MS2-binding-protein in the packaging cell line. The carrier protein (MS2-binding-domain) is expressed as a fusion protein with the di-/multi-merisation domain. The complex of MS2-binding-domain and RNA of interest formed inside the cell are actively located to the plasma membrane via the A/C Heterodimerizer compound where microvesicle formation occurs. The formed microvesicles contain a specific RNA of interest which can be delivered into target cells.

Other RNA binding domains besides the MS2 domain may also be used for RNA delivery applications, applying the same principal idea. One example of a RNA binding protein is PUMILIO1, which specifically binds a 16 nucleotide long RNA fragment (Ozawa et al., Nat Methods, 2007, 4:413-419). A second example is an arginine-rich peptide derived from the phage lambda N protein, lambdaN22, which binds a unique minimal RNA motif and can be used to tag any RNA molecule (Daigle and Ellenberg, Nat. Methods 2007, 4:633-636).

Many RNA binding proteins have a function in splicing, regulating translation or localization. However, their structure often is separated into a RNA binding domain and the actual functional domain, like a splicing domain for example. It is feasible to use functional mutants of such RNA binding proteins for specific RNA targeting purposes. For example, the SRSF1 deletion mutants containing the protein RNA binding domains (RBDs) but not the arginine serine rich activator domain (Paz et al, J. Virol. 2015; 89: 6275-6286) may be used.

Examples for RNA delivery include the delivery of mRNAs encoding for proteins involved in induction or inhibition of apoptosis in the target cells such as Cytochrome C, Bid, Bax, p53 etc; mRNAs encoding for protein factors like Oct4, Sox, cMyc, Klf4 or others known to induce the de-differentiation of somatic cells into pluripotent stem cells, or other applications. This could be achieved, for example, by expressing the RNA(s) of interest containing the stem-loop sequence together with the MS2 protein, tagged with an second dimerization domain that causes the complex of MS2 domain with the RNA(s) of interest to be localized to the inner cell membrane of the cell, where the formation of the microvesicles occurs. The formed microvesicles now contain the RNA(s) of interest that can be delivered into target cells. The RNA may be a single RNA, multiple different RNA(s), bicistronic RNA(s) containing for example an IRES sequence or a P2A (or similar) sequence. The delivery of other types of RNA includes, but not limited to, long non-coding RNA, miRNA, and shRNA/siRNA.

The same, protein-based approach could be applied to DNA delivery as well, by using DNA binding proteins and using them to bind a DNA of interest in a packing cell where the complex would be loaded into forming microvesicles. For example, the delivery of a DNA expression cassette containing a promoter followed by a DNA sequence of interest, unmodified or modified DNA oligos, like biotinylated oligos or oligos modified with other moieties. In one embodiment, the solute is DNA and the carrier protein is a DNA binding protein. The DNA binding protein, for example, may be the tet repressor (TetR), which is a prokaryotic repressor and binds tightly to a well-known operator sequence; or Zinc Fingers (ZFPs), including those particularly engineered as described in for e.g. U.S. Pat. No. 6,534,261. The delivery of a variety of different types of DNA can be envisioned.

A microvesicle dependent delivery of other, non-nucleotide cargos, like but not limited to lipids, sugars, peptides, hormones, using a cargo approach as described above can be envisioned as well.

In one embodiment, the solute is carbohydrate and the carrier protein is a lectin,

In one embodiment, the solute is ribosomes and mitochondria, which binds to their respective carrier proteins.

In the microvesicle, the membrane-associated protein and the carrier protein are bound to each other through the first and the second dimerization domain and form a multimerized complex, in which n=2-10, preferably 2-3, 2-4, or 2-5 in the complex. For example, the complex may be a dimer, trimer, or a tetramer.

In one embodiment, the first and second dimerization domains are bound to each other by a dimerization mediator. The dimerization mediator is optionally modifiable. The microvesicle may further comprises a microvesicle inducer. The membrane-associated protein, the first and second dimerization domain, the dimerization mediator, and the microvesicle inducer are similar to those described above in the first aspect of the invention.

The first and second dimerization domains may be homodimeric or heterodimeric. Heterodimeric is preferred. The first and second dimerization domains may be selected from DmrA and DmrC domains, DmrB domains, DmrD domains, dimerization domains of the dihydrofolate reductase system, dimerization domains of TAg and p53, and dimerization domains of SH2 and a PTRK protein.

In one preferred embodiment, the membrane-associated protein is selected from the group consisting of a myristoylated protein, a farnesylated protein, a membrane anchor protein, a transmembrane protein, and membrane lipid protein.

In one preferred embodiment, the microvesicle inducer is selected from the group consisting of a viral membrane fusion protein, a chemical inducer, proteolipid protein PLP1, the clathrin adaptor complex AP1, floppase, flippase scramblase, TSAP6, and CHMP4C. For example, the microvesicle inducer is a viral membrane fusion protein such as VSV-G.

The present invention provides a method of preparing a microvesicle containing a solute of interest. The method comprising: (a) maintaining a packaging cell comprising: (i) a membrane-associated protein comprising at least one first dimerization domain, (ii) a carrier protein comprising at least one second dimerization domain, and (iii) a solute that binds to the carrier protein, and (b) producing a microvesicle that contains the solute from the packaging cell under sufficient conditions, wherein the solute is selected from the group of: protein, DNA, RNA, carbohydrate, ribosomes, mitochondria, and small molecules.

In the method, the microvesicle comprises a complex of the membrane-associated protein, the carrier protein, and the solute, wherein the membrane-associated protein and the carrier protein are bound to each other, directly or indirectly, through the first and the second dimerization domains.

In the method, the packaging cell may further comprise a dimerization mediator that binds to the first and the second dimerization domains. The packaging cell may also comprise a microvesicle inducer.

In one embodiment, the packaging cell further comprises: a first expression cassette comprising a first coding sequence for encoding the membrane-associated protein comprising a first dimerization domain; and a second expression cassette comprising a second coding sequence for encoding the carrier protein comprising a second dimerization domain. In another embodiment, the packaging cell further comprises a third expression cassette comprising a third coding sequence for encoding the solute selected from the group of RNA, and protein.

In one embodiment, the carrier protein further comprises a nuclear localization signal.

### Utility

The microvesicles of the invention, e.g., as described above, find use in a variety of applications where the introduction of a protein or proteins of interest into a target cell is of interest. Applications of interest include, but are not limited to: research applications, diagnostic applications and therapeutic applications. Research applications of interest include, but are not limited to, genome modification, inducing pluripotency, cell differentiation, inducible expression systems, organelle targeting, apoptosis, cell cycle synchronization, and membrane loading and cell-cell interactions.

The microvesicles can be used to convey a variety of cargoes into target cells. These cargoes include, but are not limited to, proteins, e.g., recombinases, nucleases, transcription factors, cell cycle proteins, enzymes, apoptosis inducing proteins, protein hormones, receptors & kinases; nucleic acids, e.g. DNA & RNA, as well as lipids, carbohydrates, other macromolecules, and small molecules.

Target cells to which proteins may be delivered in accordance with the invention may vary widely. Target cells of interest include, but are not limited to: cell lines, HeLa, HEK, CHO, 293 and the like, Mouse embryonic stem cells, human stem cells, mesenchymal stem cells, primary cells, tissue samples and the like. The cells may be of various types, e.g. clonal cell lines, isolated cells, tissues, slices, and organs as well as being from various organisms, e.g. mammalian, insect, and yeast.

### Kits

Aspects of the invention further include kits, where the kits include one or more components employed in methods of making protein enriched microvesicles, e.g., as described above. In some instances, the kits may include genetic constructs which can be used to make a microvesicle producing cell. Such genetic constructs may include a coding sequence for the membrane-associated protein, e.g., as described above, where the coding sequence may be present in an expression cassette, where the promoter of the expression cassette may or may not be inducible. Genetic constructs present in the kit may include a coding sequence for the nuclease protein, e.g., as described above, where the coding sequence may be present in an expression cassette, where the promoter of the expression cassette may or may not be inducible. In yet other embodiments, the genetic construct provided in the kit may be an expression cassette configured to receive a coding sequence for a nuclease protein, but that lacks the nuclease protein coding sequence. For example, the genetic construct may include a promoter separated from a dimerization domain by a restriction site (e.g., a multiple cloning site). Genetic constructs present in the kit may include a coding sequence for the microvesicle inducer, e.g., as described above, where the coding sequence may be present in an expression cassette, where the promoter of the expression cassette may or may not be inducible. The genetic constructs may be present on separate vectors, as desired, or may be combined onto a single vector, where vectors of interest include, but are not limited, plasmids, viral vectors, etc. In some instances, the kits include microvesicle producing cells, e.g., as described above. Where desired, the kits may include additional reagents, such as microvesicle inducers, dimerization mediators, dimerization disruptors, etc. The various components of the kits may be present in separate containers, or some or all of them may be pre-combined into a reagent mixture in a single container, as desired. Reagents may also be provided in a lyophilized format—ready to use. Kits may also include a quantity of control microvesicles expressing a detectable protein (e.g., AcGFP, etc) as a control. Where desired, the kits may also include antibodies for detecting the microvesicles, substrates for quantifying microvesicle containing reporter proteins like luciferase or transfection reagents or purification systems or kits for purifying of concentrating the microvesicles.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another form of these instructions is a computer readable medium, e.g., diskette, compact disk (CD), portable flash drive, Hard Drive etc., on which the information has been recorded. Yet another form of these instructions that may be present is a website address which may be used via the internet to access the information at a removed site.

The invention is illustrated further by the following examples that are not to be construed as limiting the invention in scope to the specific procedures described in them.

### EXAMPLES

### Example 1

### Ligand Induced Dimerization/Multimerization Increases the Cargo Load in Microvesicles of the Invention

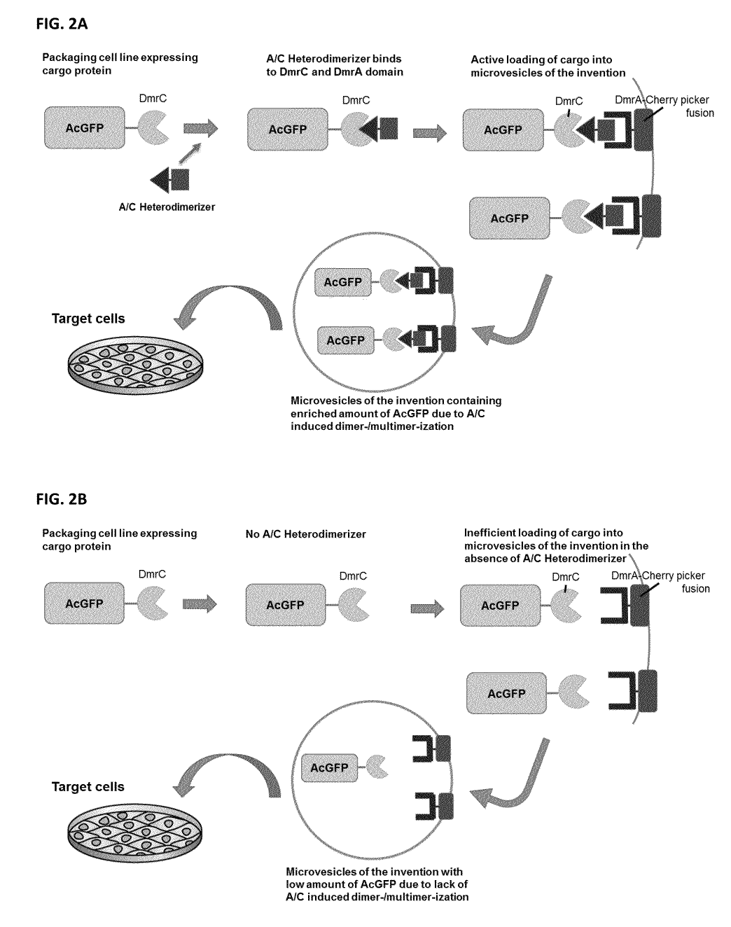

To show the increase in microvesicle packaging efficiency using ligand induced dimer-/multimer-ization, two different sets of microvesicles were prepared. The first set was produced following the production scheme shown in FIG. 2A with A/C heterodimerizer (Clontech), while a second set of microvesicles, was produced without the addition of the A/C heterodimerizer component throughout any production step. Therefore no active targeting of the AcGFP-DmrC fusion protein into microvesicles of the invention would occur (FIG. 2B).

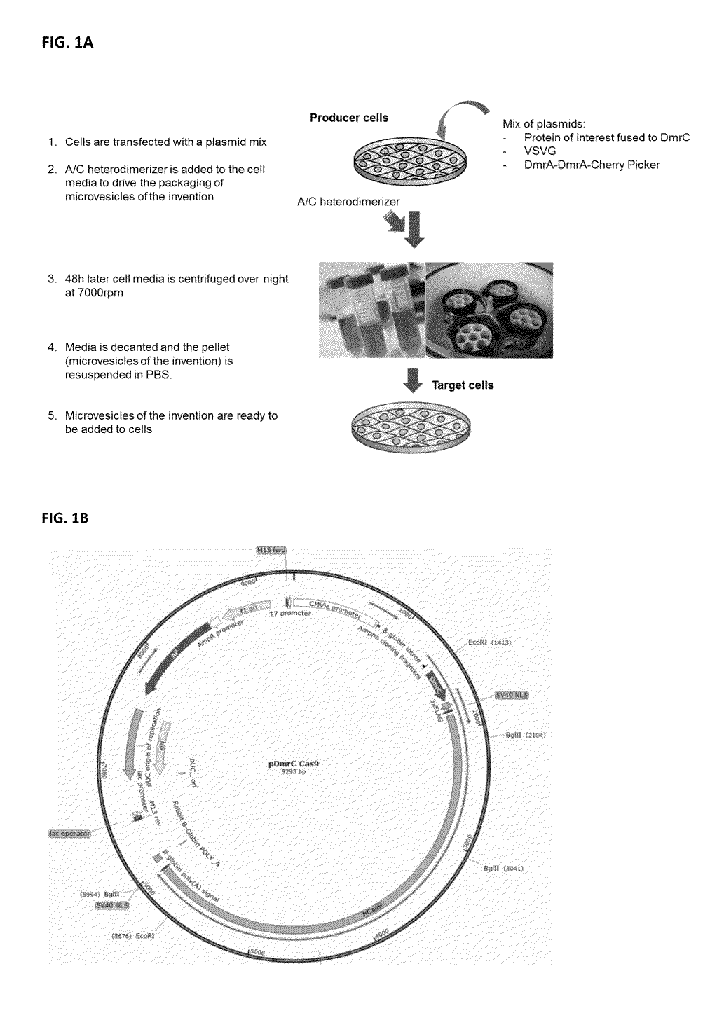

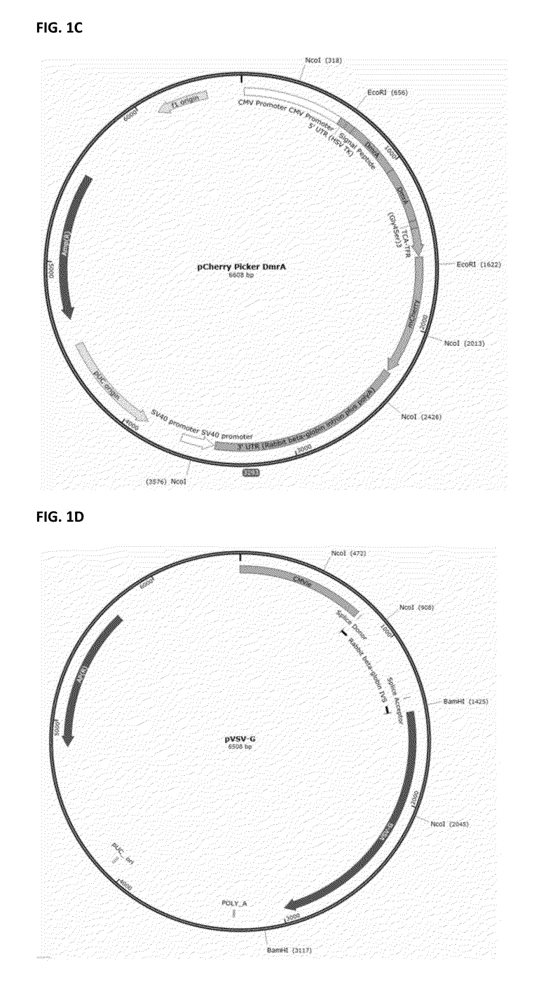

The microvesicles were generated according to the following protocol, which is outlined schematically in FIG. 1. Two sets of 4.5×10E6 HEK293T packaging cells were each plated onto a 10 cm tissue culture plate and incubated at 37° C. overnight in a tissue culture incubator following standard tissue culture procedures. 24 hours later, the A/C heterodimerizer compound was added to the media of one plate of the HEK293T packaging cell line at a final concentration between 83-500 nM. Both plates of packaging cells were then transiently co-transfected with three plasmids: 5.5 μg of pVSVG (for expression of VSV-G envelope protein); 2 μg of pCP-DmrA (for expression of the CherryPicker DmrA chimeric protein); and 22.5 μg of a third plasmid expressing AcGFP fused in-frame to a DmrC dimerization/multimerization domain. The transfection of the plasmid mix into the packaging cells can be performed using any standard transfection reagent, such as XFect, Fugene or Lipofectamine following their respective transfection protocols. 24 hours later, the A/C heterodimerizer compound was added again to one of the culture plates at a final concentration between 83-500 nM to maintain the presence of the A/C heterodimerizer in the media, allowing for continuous loading of AcGFP into the forming microvesicles.

Two to three days after the transfection, the supernatent containing the microvesicles was collected and treated with DNase1 (1.9 uL of 5 U/μL DNase1 at 37 C for 30 minutes) to remove any trace of plasmid DNA that could remain in the media. After the DNase treatment, the media containing the microvesicles was spun briefly at 500 g for 10 min to pellet any cell debris. The collected supernatant was then filtered through a 0.45 μm PVDF filter to eliminate any cells.

Finally, the microvesicle containing filtrate was centrifuged at −8000×g at 4° C. for 16 hrs using a swinging bucket rotor. After the overnight centrifugation, the supernatant was discarded, and the pelleted microvesicles resuspended in 60 to 100 μl of PBS (phosphate buffer saline pH 7.5).

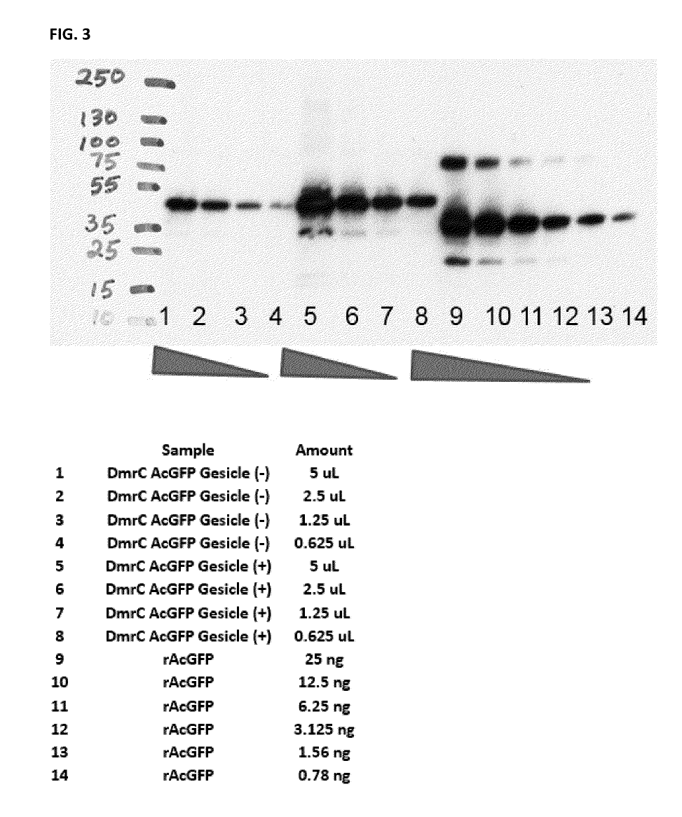

The amount of AcGPF-DmrC in the two different microvesicle preparations illustrated in FIGS. 2A and 2B was analyzed via westernblot (as shown in FIG. 3) using an anti-AcGFP monoclonal antibody. Varying amounts (0.625-5 μl) of the two microvesicle preparations, produced either with or without the A/C heterodimerizer, were separated via SDS gel electrophoresis and analyzed via westernblot analysis. A dilution series of recombinant AcGFP control protein (1.56 ng-25 ng) was run on the gel as a means to determine relative protein amounts.

The known amounts of recombinant AcGFP loaded onto the gel allowed quantification of the amount of AcGFP-DmrC protein contained in the microvesicle samples. The “−A/C” samples contained ˜0.6 ng/μl (comparing the intensity of the bands between lanes 3 and 14 in FIG. 3). However, the “+A/C” samples contained −5 ng/μl AcGFP (comparing the intensity of the bands between lanes 8 and 12 in FIG. 3). This equates to an approximate 8-fold increase in packaging efficiency of AcGFP-DmrC into microvesicles produced in the presence of the dimer-/multimer-ization agent.

### Example 2

### Delivery of Cre-Recombinase Protein Via Microvesicles of the Invention

Active packaging of the protein of interest into microvesicles of the invention by the packaging cell is especially important for proteins containing a subcellular localization sequence, like for example a nuclear localization sequence (NLS). When a protein having an NLS is expressed, it will ordinarily be translocated into the nucleus of the packaging cell line, away from the site of microvesicle formation along the plasma membrane. Using the dimer-/multimer-ization process of the invention, this effect of the NLS can be counteracted—driving translocation of the protein of interest to the site of microvesicle production. This was shown by packaging Cre-recombinase into microvesicles of the invention either in the absence or the presence of the A/C heterodimerizer compound.

Cre-recombinase recombines a pair of short target sequences called the Lox sequences. The Cre enzyme and the original Lox site called the LoxP sequence are derived from bacteriophage P1. Any DNA sequence, flanked by these LoxP sites will be precisely excised via Cre-recombinase, and the generated free DNA ends will be re-joined.

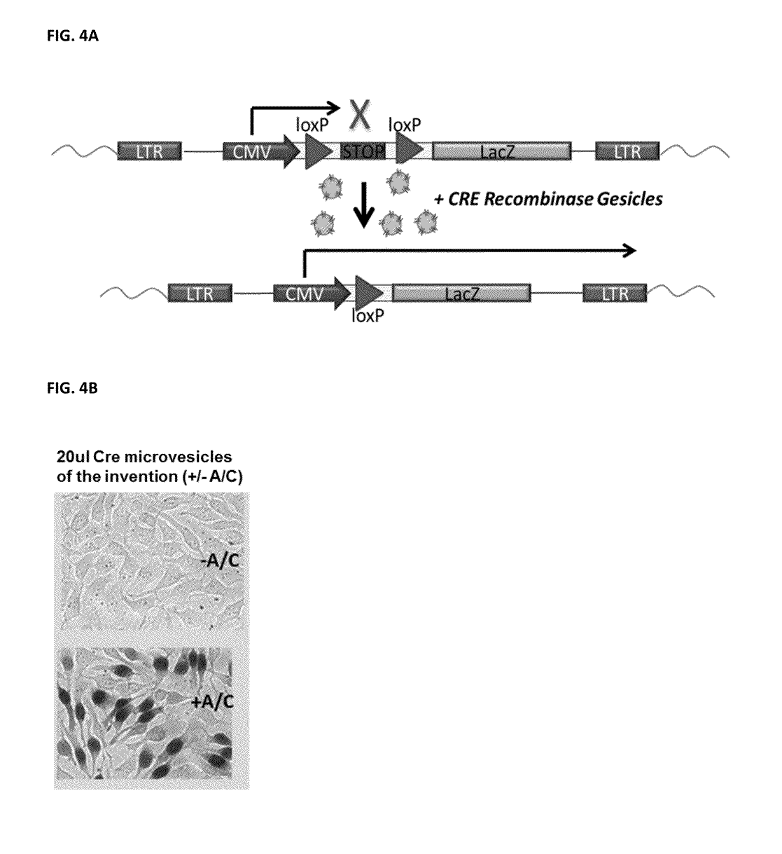

A commercially available rhabdomyosarcoma cell line harboring an integrated, loxP-conditional LacZ expression cassette was used to investigate the advantage of active loading of Cre-DmrC-NLS into microvesicles of the invention (FIG. 4A). Successful delivery of Cre-recombinase to this cell line will cause the excision of the stop codon flanked by loxP sites in the LacZ gene, resulting in the expression of the full-length, functional LacZ gene product, beta-galactosidase. The presence of this enzyme in cells can then be detected via the substrate X-Gal, turning cells blue upon cleavage of the substrate by the LacZ enzyme.

Microvesicles of the invention were produced following the microvesicle production protocol outlined in Example 1 by transfecting the packaging cells with the microvesicle packaging mix (containing the VSV-G expressing plasmid and the Cherry Picker-DmrA expressing plasmid) and a plasmid encoding for Cre-DmrC-NLS. The microvesicle production was performed following the standard microvesicle production protocol as outlined in Example 1 either in the presence or the absence of A/C heterodimerizer in the media.

The two different Cre microvesicle preparations were then separately added to the rhabdomysarcoma cell line. Cells treated with Cre-microvesicles produced in the absence of A/C heterodimerizer (−A/C) compound showed no detectable beta-galactosidase activity. However, cells treated with Cre-microvesicles produced in the presence of A/C heterodimerizer compound (+A/C) showed blue staining of many cells, indicating the expression of full length beta galactosidase due to the excision of the stop codon by the Cre-recombinase, successfully delivered via microvesicles of the invention (FIG. 4B).

### Example 3

### Delivery of Functional Cas9 Via Microvesicles of the Invention for Genome Editing Applications

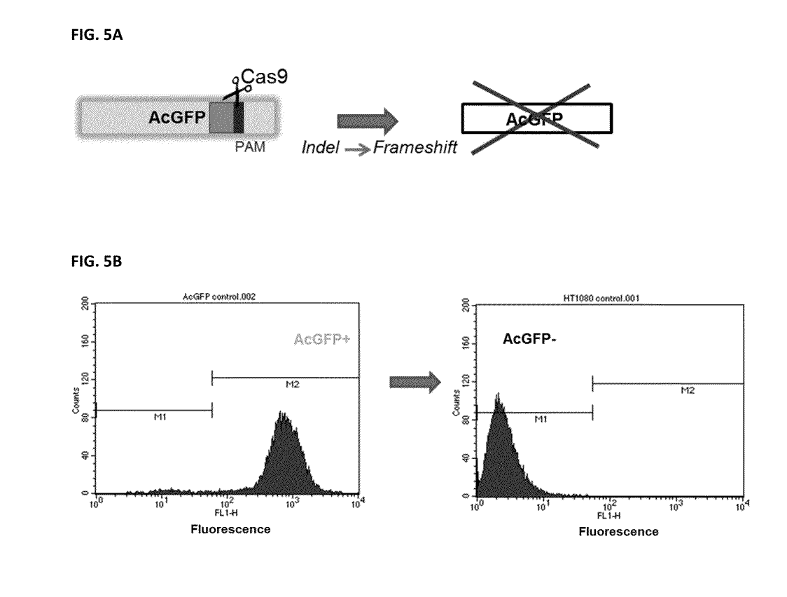

The ability to deliver Cas9 via microvesicles of the invention was first evaluated on an HT1080 cell line engineered to contain a stably integrated, single copy of an AcGPF green fluorescent protein expression cassette. In this model system, the AcGFP expression cassette was targeted via Cas9/sgRNA, so as to cause insertions or deletions (indels) at the site of the double strand break created by the sgRNA targeted Cas9 endonuclease (FIG. 5A). Flow cytometer analysis for the green fluorescence signal allowed a simple read-out to determine the knockout efficiency in the targeted cell population (FIG. 5B).

Microvesicles of the invention were produced in the presence of the A/C heterodimerizer compound, following the standard microvesicle production protocol outlined in example 1 by co-transfecting the packaging cells with the microvesicle packaging mix and a plasmid encoding for Cas9-NLS-DmrC.

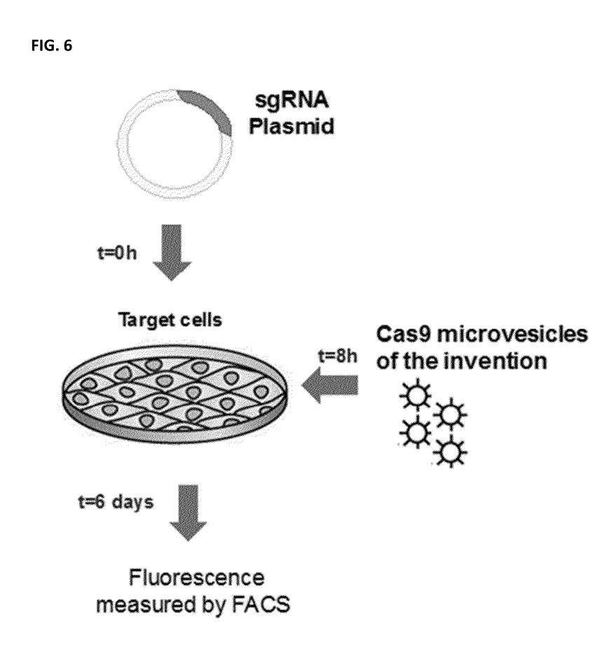

The HT1080-AcGFP cell line was first transfected with a plasmid encoding for an sgRNA targeting the AcGFP gene. 8 hours after the transfection, the cells were treated with the Cas9-DmrC-NLS containing microvesicles of the invention following the standard microvesicle treatment protocol (FIG. 6).

Six days after microvesicle treatment, target cells were analyzed via flow cytometry to determine the AcGFP knockout efficiency obtained via delivery of Cas9 protein via microvesicles of the invention.

The flow cytometry data of the parental HT1080-AcGFP cell line showed a single cell population exhibiting green fluorescence (FIG. 7A).

However, HT1080-AcGFP cells, transiently transfected with a plasmid encoding an sgRNA targeting AcGFP and treated with Cas9-NLS-DmrC microvesicles of the invention, appear in two distinct populations. About 61% of the cell population had lost its green fluorescence due to the AcGFP gene being knocked out by the sgRNA targeted Cas9-NLS-DmrC (FIG. 7B).These data support the use of Cas9 protein delivery via microvesicles of the invention as a method to perform successful gene editing.

### Example 4

### Optimization of the sgRNA

The single guide RNA (sgRNA) contains two different functional domains. The actual guiding domain, responsible for binding to the target DNA sequence and the scaffold domain, responsible for loading the sgRNA onto the Cas9 endonuclease.

Two different scaffold sgRNA sequences (FIG. 8A: SEQ ID NO: 1 and FIG. 8B: SEQ ID NO: 2) were tested to determine potential functional differences in creating Cas9-mediated indels on a target sequence.

The two different sgRNA scaffolds were tested by performing the same experiment outlined in Example 3 and shown in FIG. 6 and FIG. 7.

The HT1080-AcGPF cell line was transfected with a plasmid encoding for a sgRNA targeting AcGFP designed using either scaffold 1 (SEQ ID. 1) or scaffold 2 (SEQ ID 2). In both cases the Cas9 was delivered via microvesicles of the invention using the standard microvesicle treatment protocol.

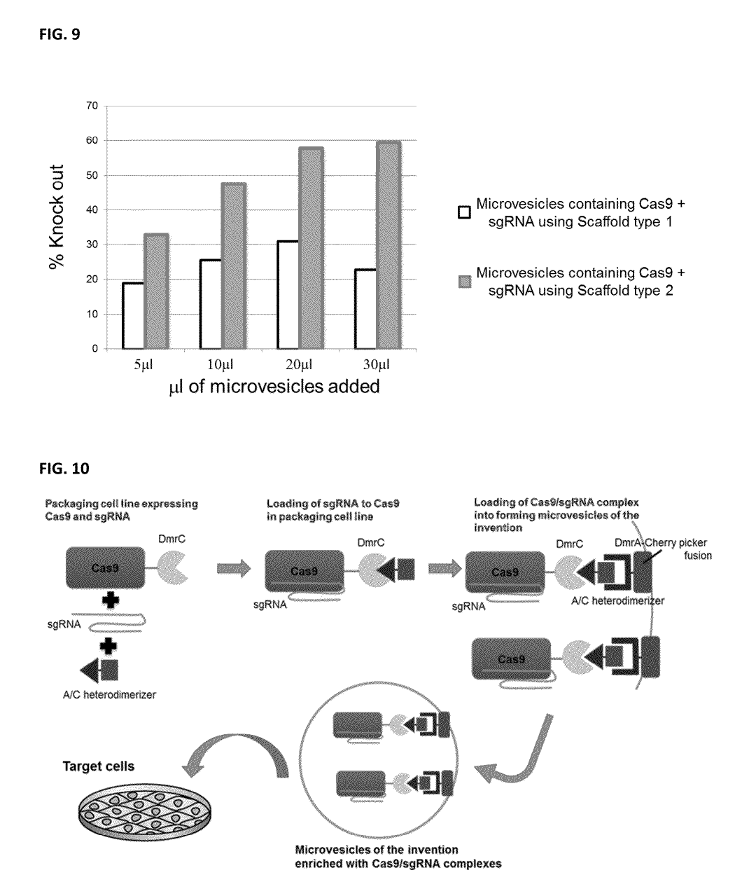

As shown in FIG. 9, sgRNAs designed using scaffold 2 achieved consistently higher target knockout efficiencies as compared to sgRNAs designed using scaffold 1.

### Example 5

### Delivery of Functional Cas9/sgRNA Complexes Via Microvesicles of the Invention for Genome Editing Applications

In the previous examples, Cas9 endonuclease was packaged into the microvesicles of the invention using the A/C heterodimerizer compound, in the absence of the sgRNA, which was provided to the target cell separately. To deliver both Cas9 as well as the sgRNA as a protein/RNA complex using microvesicles, the packaging cell line was transfected with both the Cas9-DmrC-NLS encoding vector as well as an sgRNA encoding expression vector. Therefore both were expressed in the packaging cell line. The Cas9 protein then acts as a carrier protein—carrying the sgRNA into the microveicles. This resulted in the production of microvesicles preloaded with the sgRNA of interest and Cas9 (FIG. 10). Microvesicle production was performed following the microvesicle production protocol out-lined in Example 1 except that 10 ug of sgRNA plasmid and 22.5 μg of Cas9 plasmid were used together with 5.5 μg of VSVG and 2 μg of cherry picker.

By expressing both, Cas9-NLS-DmrC and the sgRNA in the packaging cells, the sgRNA is loaded onto the Cas9 protein in the packaging cell line. This protein/RNA complex is then actively loaded into forming microvesicles of the invention via the A/C heterodimerizer compound as shown in FIG. 10.

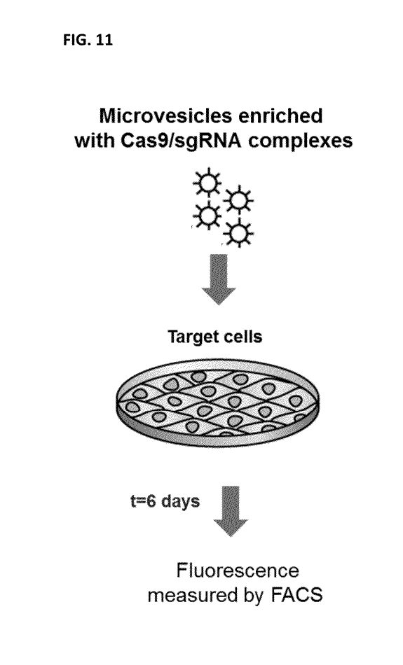

The preloaded Cas9/sgRNA microvesicles of the invention, targeting AcGFP, were delivered into HT1080-AcGFP cells using the microvesicle treatment protocol (FIG. 11).

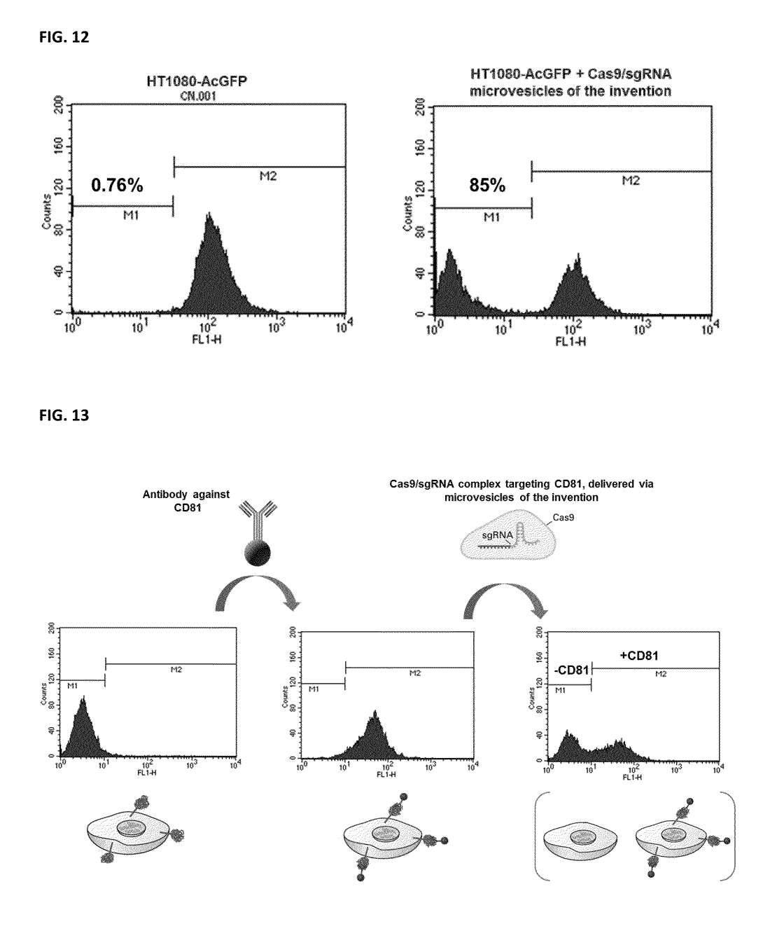

Flow cytometry analysis of the parental HT1080-AcGFP cell line showed a single cell population exhibiting green fluorescence (FIG. 12, left panel). However, HT1080-AcGFP cells, treated with microvesicles of the invention containing a Cas9/sgRNA complex targeting AcGFP showed two distinct populations when analyzed via flow cytometry (FIG. 12, right panel). About 85% of the cell population was found to have lost its green fluorescence due to the AcGFP gene being knocked out by the preformed Cas9/sgRNA complex delivered via the microvesicles.

### Example 6

### Knockout of an Endogenous Target Gene Using Microvesicles Preloaded with Cas9 and an sgRNA Targeting the Endogenous Gene

Similar results were obtained when targeting the endogenous membrane receptor, CD81 of HeLa cells via microvesicles of the invention. In this experiment, microvesicles were generated that contained Cas9/sgRNA complex wherein the complexed sgRNA targeted the CD81 encoding gene. As previously, the microvesicles were produced following the standard microvesicle production protocol, outlined in Example 1, with A/C heterodimerizer, by co-transfecting the packaging HEK293T cell line with the microvesicle packaging mix (5.5 μg of VSVG and 2 μg of cherry picker) as well as a vector expressing Cas9-DmrC-NLS (22.5 μg) and a sgRNA encoding plasmid (10 ug) targeting CD81.

500 μl of a HeLa cell suspension (6×10³ cells/ml) was plated in wells of a 24 well tissue culture plate 24 hours before microvesicle treatment. 24 hours after plating, protamine at a final concentration of 8 ug/ml was added to each well followed by the addition of 30 ul of Cas9/sgRNA microvesicles. The 24 well tissue culture plate was then centrifuged at 2500 rpm for 30 minutes to allow for increased microvesicle-delivery efficiency. After centrifugation, the plate was transferred back into the tissue culture incubator for 48 h.

The cells were expanded into a 10 cm plate upon confluency and analyzed for knockout efficiency after 5 to 6 days (depending on the half life of the target protein).

The loss of CD81 expression upon successful knockout was monitored via flow cytometry using a fluorescently labeled antibody against CD81.

Flow cytometry analysis data of the parental HeLa cell line shown in FIG. 13 show a single population that is positive for the presence of CD81 on the cell surface.

However, HeLa cells treated with the CD81-targeting Cas9/sgRNA microvesicles containing a preformed Cas9/sgRNA complex targeting CD81, appear in two distinct populations. As shown in FIG. 13, about 44% of the cell population was negative for antibody staining due to the successful knockout and loss of CD81 expression on the cell surface.

The invention, and the manner and process of making and using it, are now described in such full, clear, concise and exact terms as to enable any person skilled in the art to which it pertains, to make and use the same. It is to be understood that the foregoing describes preferred embodiments of the present invention and that modifications may be made therein without departing from the scope of the present invention as set forth in the claims. To particularly point out and distinctly claim the subject matter regarded as invention, the following claims conclude this specification.

What is claimed is:

1. A microvesicle comprising:
(i) a membrane-associated protein comprising at least one first dimerization domain,
(ii) a carrier protein comprising at least one second dimerization domain, and
(iii) a solute that binds to the carrier protein, wherein the solute is selected from the group of: DNA, RNA, protein, carbohydrate, ribosomes, mitochondria, and small molecules.

2. The microvesicle according to claim 1, wherein the solute is single guide RNA and the carrier protein is a nuclease.

3. The microvesicle according to claim 2, wherein the nuclease is CAS9, CAS9 mutant without nuclease activity, or Nickase mutant of Cas9.

4. The microvesicle according to claim 3, wherein the single guide RNA comprises the nucleic acid sequence of SEQ ID NO: 1 or 2, and the nuclease is CAS9.

5. The microvesicle according to claim 2, wherein the nuclease is Argonaute nuclease.

6. The microvesicle according to claim 1, wherein the solute is RNA and the carrier protein is a RNA binding protein.

7. The microvesicle according to claim 6, wherein the RNA binding protein is bacteriophage MS2 coat protein, lambdaN22, PUMILIO1, or SRSF1 deletion mutants.

8. The microvesicle according to claim 1, wherein the solute is DNA and the carrier protein is a DNA binding protein.

9. The microvesicle according to claim 1, wherein the membrane-associated protein is selected from the group consisting of a myristoylated protein, a farnesylated protein, a membrane anchor protein, a transmembrane protein, and membrane lipid protein.

10. The microvesicle according to claim 1, wherein the membrane-associated protein and the carrier protein are bound to each other through the first and the second dimerization domain and form a multimerized complex.

11. The microvesicle according to claim 10, wherein first and second dimerization domains are bound to each other by a dimerization mediator.

12. The microvesicle according to claim 1, wherein the microvesicle further comprises a microvesicle inducer.

13. The microvesicle according to claim 12, wherein the microvesicle inducer is selected from the group consisting of a viral membrane fusion protein, a chemical inducer, proteolipid protein PLP1, the clathrin adaptor complex AP1, floppase, flippase scramblase, TSAP6 and CHMP4C.

14. The microvesicle according to claim 1, wherein the first and second dimerization domains are selected from DmrA and DmrC domains, DmrB domains, DmrD domains, dimerization domains of the dihydrofolate reductase system, dimerization domains of TAg and p53, and dimerization domains of SH2 and a PTRK protein.

15. A method of preparing the microvesicle according to claim 1, the method comprising:
(a) maintaining a packaging cell comprising:
(i) the membrane-associated protein comprising at least one first dimerization domain,
(ii) the carrier protein comprising at least one second dimerization domain, and
(iii) the solute that binds to the carrier protein, and
(b) producing the microvesicle from the packaging cell under sufficient conditions.

16. The method according to claim 15, wherein the packaging cell further comprises:
a first expression cassette comprising a first coding sequence for encoding the membrane-associated protein comprising a first dimerization domain; and
a second expression cassette comprising a second coding sequence for encoding the carrier protein comprising a second dimerization domain.

17. The method according to claim 16, wherein the packaging cell further comprises a third expression cassette comprising a third coding sequence for encoding the solute selected from the group of RNA, and protein.

18. A microvesicle comprising:
(i) a membrane-associated protein comprising at least one first dimerization domain, and
(ii) a nuclease protein comprising at least one second dimerization domain,
wherein said nuclease is selected from the group consisting of a Cas protein, and Argonaute nuclease.

19. The microvesicle according to claim 18, wherein the Cas protein is elected from the group consisting of: Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, and modified versions thereof.

20. The microvesicle according to claim 18, wherein the membrane-associated protein and the nuclease are bound to each other through the first and the second dimerization domain and form a multimerized complex.
